(19) United States
(12) Patent Application Publication
(10) Pub. No.: US 2016/0120515 A1
(43) Pub. Date: May 5, 2016

(54) ULTRASONIC MEASUREMENT DEVICE AND ULTRASONIC IMAGING DEVICE

(71) Applicant: SEIKO EPSON CORPORATION, Tokyo (JP)

(72) Inventor: Yoshio ARAI, Shiojiri (JP)

(21) Appl. No.: 14/920,309

(22) Filed: Oct. 22, 2015

(30) Foreign Application Priority Data
Oct. 31, 2014 (JP) 2014-222471

(51) Int. Cl. A61B 8/08; A61B 8/14; A61B 8/00

(52) U.S. Cl. CPC A61B 8/5269; A61B 8/4483; A61B 8/14; A61B 8/461

(57) ABSTRACT

An ultrasonic measurement device 100 includes a pulse signal output circuit 110 that outputs a pulse signal having a rectangular wave based on a clock signal, and a resonance circuit 120 that is connected to an output node of the pulse signal output circuit 110, includes an ultrasonic transducer element, and has frequency characteristics of a low-pass filter. Also, the pulse signal output circuit 110 outputs a plurality of pulse signals that are different from each other in at least one of pulse signal voltage, pulse signal width, and pulse output timing.

### BACKGROUND

1. Technical Field

The present invention relates to an ultrasonic measurement device, an ultrasonic imaging device and the like.

2. Related Art

As a device for use in examining the inside of a human body that serves as a test subject, an ultrasonic measurement device that emits an ultrasonic wave toward the subject and receives reflected waves from interfaces of objects having different acoustic impedances inside the subject draws an attention. The ultrasonic measurement device is also applied to diagnostic imaging that is performed on a superficial layer of the test subject to measure the visceral fat, the blood flow rate, and the like.

When measurement using, for example, an ultrasonic measurement device is performed and a B-mode image is generated, it is necessary to reduce scatter noise caused when an ultrasonic echo (received wave) is received, in order to improve S/N of the received wave. Accordingly, it is preferable, for example, that a transmission signal that is to be input to the ultrasonic transducer elements of the ultrasonic measurement device be prevented from including a harmonic component, and have a short transient response as well.

JP-A-11-56839 discloses, as an invention relating to such an ultrasonic measurement device, a technique for approximating a transmission wave that is to be input to the ultrasonic transducer elements to a sine wave. Furthermore, JP-A-2010-194045 discloses a method for shortening the transient response of a transmission wave by reducing the pulse width of a rectangular wave drive pulse that is to be input to the ultrasonic transducer elements.

However, even using the above-described method of JP-A-11-56839, a transmission wave includes a harmonic component because an ordinary transmission drive waveform output from a pulser is a rectangular wave. Particularly, when using harmonic imaging, if a transmission wave includes a harmonic component, it is impossible to distinguish whether a harmonic component included in a received wave is a harmonic component caused by a nonlinear effect or a harmonic component based on the harmonic component included in the transmission wave, causing the problem that it is not possible to generate an appropriate B-mode image.

Furthermore, the above-described method of JP-A-2010-194045 has the problem that it is difficult to achieve a sufficient damping effect because a pulse voltage is constant.

### SUMMARY

According to some aspects of the invention, it is possible to provide an ultrasonic measurement device, an ultrasonic imaging device, and the like that can remove a harmonic component of a transmission wave to be input to the ultrasonic transducer elements, so as to suppress the transient response of the transmission wave.

According to an aspect of the invention, an ultrasonic measurement device includes: a pulse signal output circuit that outputs a rectangular wave pulse signal based on a clock signal; and a resonance circuit that is connected to an output node of the pulse signal output circuit, includes an ultrasonic transducer element, and has frequency characteristics of a low-pass filter, wherein the pulse signal output circuit outputs a plurality of pulse signals that are different from each other in at least one of pulse signal voltage, pulse signal width, and pulse output timing.

According to the aspect of the invention, the pulse signal output circuit outputs, to the resonance circuit, a plurality of pulse signals that are different from each other in at least one of pulse signal voltage, pulse signal width, and pulse output timing, and inputs a transmission signal based on the plurality of input pulse signals to the ultrasonic transducer element. Accordingly, it is possible to remove a harmonic component of a transmission wave to be input to the ultrasonic transducer element, thereby suppressing the transient response of the transmission wave.

Furthermore, according to the aspect of the invention, it is preferable that the pulse signal output circuit output a first pulse signal having a first pulse voltage at a first pulse output timing, and output a second pulse signal having a second pulse voltage, which is different from the first pulse voltage, at a second pulse output timing after the first pulse output timing.

Accordingly, it is possible to output pulse signals having different voltages at different timings, thereby performing control of the amplitude of the transmission wave, suppression of the transient response, and the like.

Furthermore, according to the aspect of the invention, it is preferable that the first pulse signal be a first polarity pulse signal having one of the positive polarity and the negative polarity, and the second pulse signal be a second polarity pulse signal having the other different polarity, and the absolute value of the second pulse voltage be smaller than the absolute value of the first pulse voltage.

Accordingly, it is possible, for example, to suppress an increase in the amplitude of a transmission wave that corresponds to the second pulse signal as compared with the amplitude of a transmission wave that corresponds to the first pulse signal.

Furthermore, according to the aspect of the invention, it is preferable that the pulse signal output circuit output a first pulse signal having a first pulse width at the first pulse output timing, and output a second pulse signal having a second pulse width, which is different from the first pulse width, at the second pulse output timing after the first pulse output timing.

Accordingly, it is possible to output pulse signals having different pulse widths at different timings, and to perform control of the amplitude of the transmission wave, suppression of the transient response, and the like.

Furthermore, according to the aspect of the invention, it is preferable that the second pulse width be greater than the first pulse width.

Accordingly, it is possible, for example, to suppress an increase in the amplitude of the transmission wave on the positive polarity side due to resonant vibration after the amplitude of the transmission wave shows a negative value due to the second pulse signal, thereby suppressing the transient response.

Furthermore, according to the aspect of the invention, it is preferable that the second pulse signal be a pulse signal for suppressing resonant vibration of a transmission signal to the ultrasonic transducer element.

Accordingly, it is possible, for example, to suppress resonant vibration of a transmission signal.

Furthermore, according to the aspect of the invention, it is preferable that the second pulse signal be a pulse signal for suppressing sound reverberation of a transmission signal to the ultrasonic transducer element.

Accordingly, it is possible, for example, to suppress sound reverberation (transient response) of a transmission signal.

Furthermore, according to the aspect of the invention, it is preferable that the pulse signal output circuit output the first pulse signal having the first pulse voltage and the first pulse width at the first pulse output timing, and output, at the second pulse output timing after the first pulse output timing, the second pulse signal having the second pulse voltage whose absolute value is smaller than that of the first pulse voltage and having the second pulse width, which is greater than the first pulse width.

Accordingly, it is possible, for example, to suppress an increase in the amplitude of the transmission wave corresponding to the second pulse signal as compared with the amplitude of the transmission wave corresponding to the first pulse signal, and to suppress an increase in the amplitude of the transmission wave on the positive polarity side due to resonant vibration after the amplitude of the transmission wave shows a negative value due to the second pulse signal, thereby suppressing the transient response.

Furthermore, according to the aspect of the invention, it is preferable that the pulse signal output circuit output one or more first time period pulse signals during a first time period, output no pulse signals during a second time period after the first time period, and output a third time period pulse signal during a third time period after the second time period.

Accordingly, it is possible, for example, to approximate an envelope curve of a transmission waveform to a (substantial) sine wave curve using simple timing control of rectangular wave driving, without increasing the number of voltage supplies, thereby shortening the transient response of the transmission wave.

Furthermore, according to the aspect of the invention, it is preferable that the pulse signal output circuit outputs a pulse signal for suppressing sound reverberation of a transmission signal to the ultrasonic transducer element during the third time period.

Accordingly, it is possible, for example, to suppress sound reverberation (transient response) of a transmission signal.

Furthermore, according to the aspect of the invention, it is preferable that the pulse signal output circuit output a pulse signal that causes an envelope curve of a waveform of a transmission signal to the ultrasonic transducer element to have the shape of a sine wave.

Accordingly, when performing, for example, harmonic imaging, appropriate image generation and the like is possible only using a harmonic component caused by a nonlinear effect because there is no reflected wave due to a harmonic component included in the transmission wave.

Furthermore, according to another aspect of the invention, an ultrasonic imaging device includes the ultrasonic measurement device, and a display unit that displays image data for display that is created based on an ultrasonic echo in response to a transmitted ultrasonic wave.

### BRIEF DESCRIPTION OF THE DRAWINGS

The invention will be described with reference to the accompanying drawings, wherein like numbers reference like elements.

FIGS. 1A and 1B illustrate examples of a configuration of a transmission circuit of an ultrasonic measurement device according to the present embodiment.

FIG. 2 illustrates an example of a configuration of a pulser.

FIG. 3 is a diagram illustrating a method for driving the pulser according to a first embodiment.

FIG. 4 is a diagram illustrating a pulser output waveform and a transmission waveform according to the first embodiment.

FIG. 5 is a diagram illustrating a method for driving the pulser according to a first example of the first embodiment.

FIG. 6 is a diagram illustrating a pulser output waveform and a transmission waveform according to the first example of the first embodiment.

FIG. 7 illustrates another example of a configuration of the pulser.

FIG. 8 is a diagram illustrating a method for driving the pulser according to a second example of the first embodiment.

FIG. 9 is a diagram illustrating a pulser output waveform and a transmission waveform according to the second example of the first embodiment.

FIG. 10 is a diagram illustrating a method for driving the pulser according to a third example of the first embodiment.

FIG. 11 is a diagram illustrating a pulser output waveform and a transmission waveform according to the third example of the first embodiment.

FIG. 12 is a diagram illustrating a method for driving the pulser according to a fourth example of the first embodiment.

FIG. 13 is a diagram illustrating a pulser output waveform and a transmission waveform according to the fourth example of the first embodiment.

FIG. 14 is a diagram illustrating a method for driving the pulser according to a fifth example of the first embodiment.

FIG. 15 is a diagram illustrating a pulser output waveform and a transmission waveform according to the fifth example of the first embodiment.

FIG. 16 is a diagram illustrating a method for driving the pulser according to a sixth example of the first embodiment.

FIG. 17 is a diagram illustrating a pulser output waveform and a transmission waveform according to the sixth example of the first embodiment.

FIGS. 18A to 18C illustrate transmission waveforms having a half cycle of wave.

FIG. 19 is a diagram illustrating a method for driving the pulser according to a first example of a second embodiment.

FIG. 20 is a diagram illustrating a pulser output waveform and a transmission waveform according to the first example of the second embodiment.

FIG. 21 is a diagram illustrating a method for driving the pulser according to a second example of the second embodiment.

FIG. 22 is a diagram illustrating a pulser output waveform and a transmission waveform according to the second example of the second embodiment.

FIG. 23 is a diagram illustrating a method for driving the pulser according to a third example of the second embodiment.

FIG. 24 is a diagram illustrating a pulser output waveform and a transmission waveform according to the third example of the second embodiment.

FIG. 25 is a diagram illustrating a method for driving the pulser according to a fourth example of the second embodiment.

FIG. 26 is a diagram illustrating a pulser output waveform and a transmission waveform according to the fourth example of the second embodiment.

FIGS. 27A to 27C illustrate examples of a configuration of an ultrasonic transducer element.

FIG. 28 illustrates an example of a configuration of an ultrasonic transducer device.

FIGS. 29A and 29B illustrate examples of a configuration of an ultrasonic transducer element group that is provided for each channel.

FIGS. 30A to 30C illustrate examples of a configuration of an ultrasonic imaging device according to the present embodiment.

### DESCRIPTION OF EXEMPLARY EMBODIMENTS

Hereinafter, embodiments of the invention will be described. Note that the embodiments that will be described below do not unduly limit the content of the invention recited in Claims. Furthermore, all configurations that will be described in the embodiments are not necessarily essential components of the invention.

### 1. Overview

As described above, when, for example, measurement using an ultrasonic measurement device is performed and a B-mode image is generated, it is necessary to reduce scatter noise caused when an ultrasonic echo (received wave) is received, in order to improve S/N of the received wave. Accordingly, it is preferable, for example, that a transmission signal (transmission wave) that is to be input to the ultrasonic transducer elements of the ultrasonic measurement device be prevented from including a harmonic component, and have a short transient response as well. Furthermore, it is preferable that the absolute value of the amplitude of the transmission wave on the positive polarity side and the absolute value of the amplitude of the transmission wave on the negative polarity side be equal to each other.

However, in the above-described method of JP-A-11-56839, a transmission wave includes a harmonic component because an ordinary transmission drive waveform output from a pulser is a rectangular wave. Particularly, when using harmonic imaging, if a transmission wave includes a harmonic component, it is not possible to distinguish whether a harmonic component included in the received wave is a harmonic component caused by a nonlinear effect or a harmonic component based on the harmonic component included in the transmission wave, and an appropriate B-mode image cannot be generated. Furthermore, also in the above-described method of JP-A-2010-194045, it is difficult to achieve a sufficient damping effect because the pulse voltage is constant.

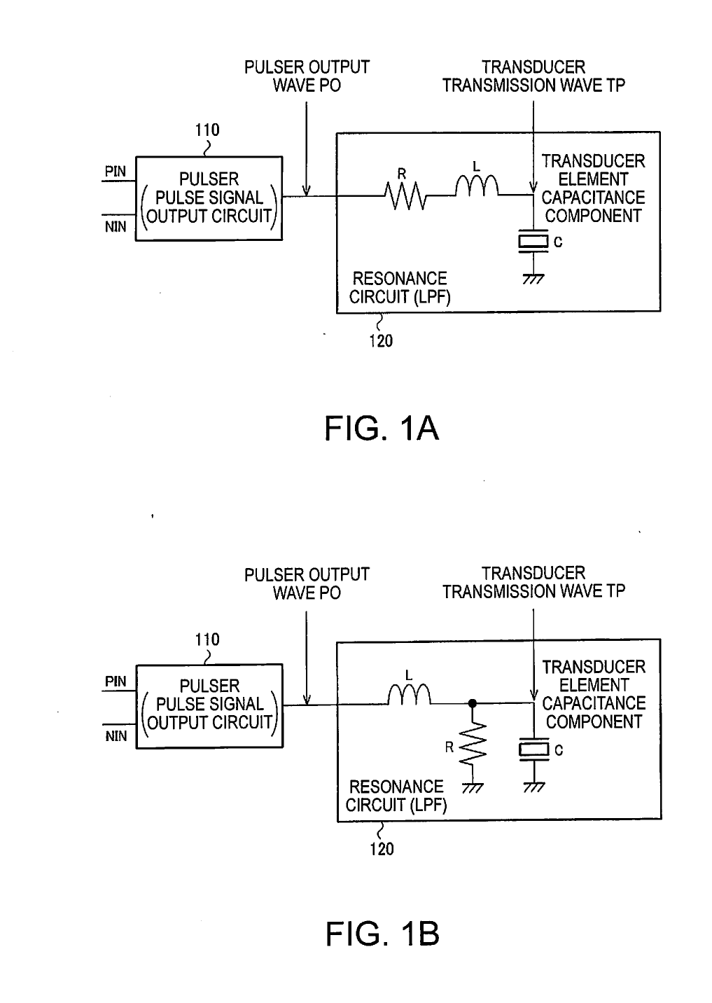

Accordingly, as shown in FIG. 1A or 1B, an ultrasonic measurement device 100 according to embodiments that will be described below includes a pulse signal output circuit (pulser) 110 that outputs a pulse signal of a rectangular wave based on a clock signal, and a resonance circuit 120 that is (electrically) connected to an output node of the pulse signal output circuit 110, includes an ultrasonic transducer element, and has frequency characteristics of a low-pass filter (LPF).

Also, the pulse signal output circuit 110 outputs a plurality of pulse signals that are different from each other in at least one of pulse signal voltage, pulse signal width, and pulse output timing.

That is, in the embodiments, the pulse signal output circuit 110 outputs, to the resonance circuit 120, a plurality of pulse signals that are different from each other in at least one of pulse signal voltage, pulse signal width, and pulse output timing, and inputs, to the ultrasonic transducer elements, a transmission signal based on the plurality of pulse signals input to the resonance circuit 120. In other words, the pulse voltages, the pulse widths, and the pulse output timings at the time of rectangular wave driving are controlled to obtain a transmission wave of a (substantial) sine wave that has a short transient response. Accordingly, it is possible to remove a harmonic component of a transmission wave that is to be input to the ultrasonic transducer elements, and to suppress the transient response of the transmission wave.

### 2. First Embodiment

### 2.1. System Configuration Example

Hereinafter, FIGS. 1A and 1B illustrate examples of a configuration of a transmission circuit included in the ultrasonic measurement device 100 of the present embodiment. The transmission circuit shown in FIGS. 1A and 1B includes a pulser 110 (pulse signal output circuit 110), and a low-pass filter on the output side of the pulser 110. Furthermore, as described above, this low-pass filter constitutes, together with the ultrasonic transducer elements (vibrating elements), the resonance circuit 120.

FIG. 1A illustrates an example in which the low-pass filter LCR is configured by providing passive elements, which are an inductor L and a resistor R, in series to the ultrasonic transducer elements having a capacitance component C. The capacitance component C of the ultrasonic transducer elements also serves as a constituent component of the low-pass filter. Furthermore, in view of configuring the low-pass filter, it is also possible to provide the passive capacitance elements in parallel to the ultrasonic transducer elements, but this configuration is omitted in the present example for ease of description.

On the other hand, FIG. 1B illustrates an example in which the low-pass filter is configured by connecting the ultrasonic transducer elements and the inductor L in series and connecting the ultrasonic transducer elements and the resistor R in parallel. Both configurations shown in FIGS. 1A and 1B have the same low-pass filter function. Note that the ultrasonic measurement device 100 is not limited to the configurations of FIGS. 1A and 1B, and various modifications, such as one in which some of the constituent components is omitted or one in which another constituent component is added, can be executed.

Furthermore, as shown in FIGS. 1A and 1B, a pulser output wave PO is a signal that is output from the pulser 110 and is to be input to the resonance circuit 120. Furthermore, a transmission wave TP is a signal that is to be input to the ultrasonic transducer elements based on the pulser output wave PO.

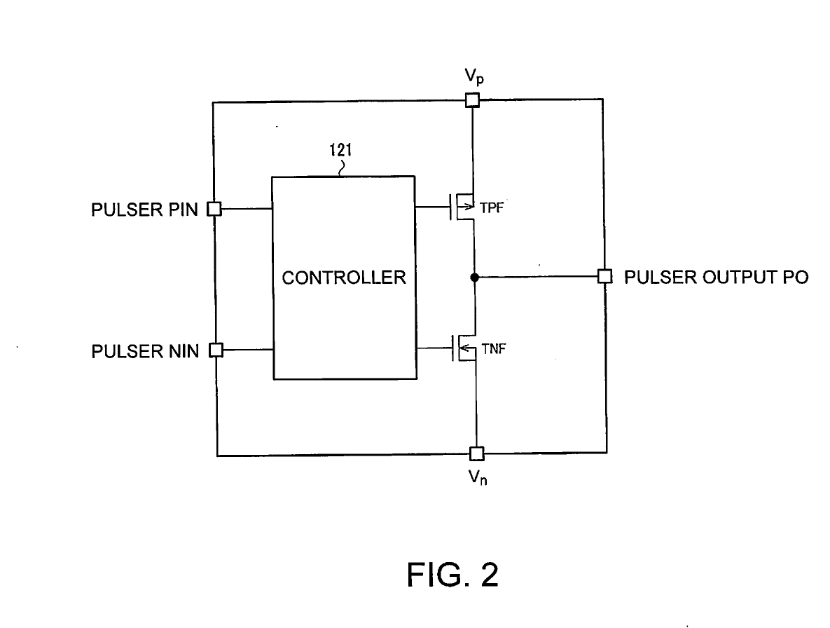

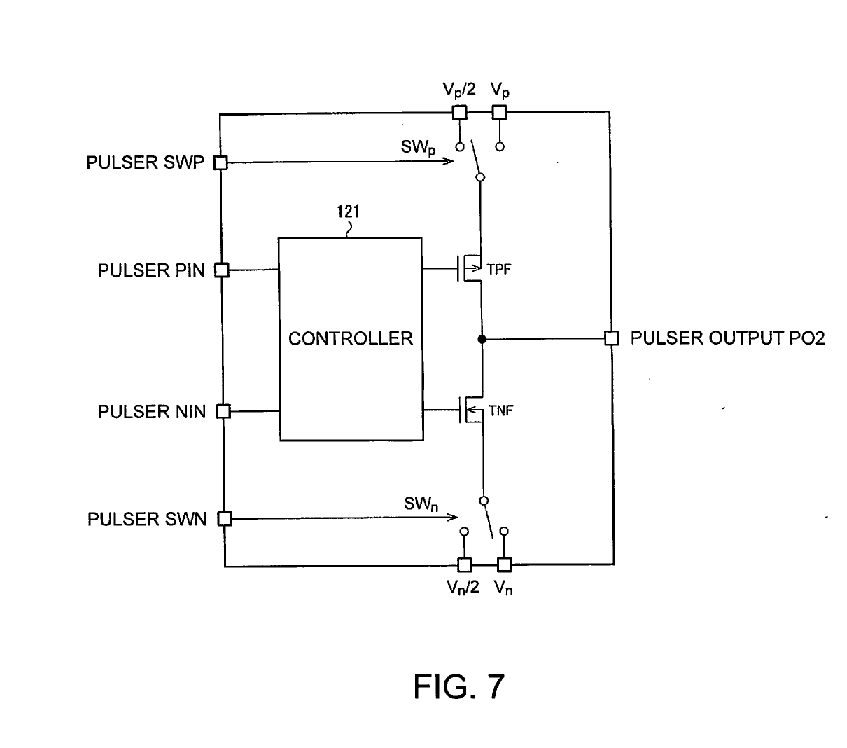

FIG. 2 is a diagram illustrating a configuration of the pulser 110. The pulser 110 includes a P-type MOSFET (TPF) switch element that corresponds to a positive power-supply voltage Vₚ, an N-type MOSFET (TNF) switch element that corresponds to a negative power-supply voltage Vₙ, and a controller 121. Gate trigger signals of the P-type MOSFET (TPF) and the N-type MOSFET (TNF) are driven and controlled by a drive control signal (logic signal) PIN and a drive control signal NIN via the controller 121 to form a positive pulse and a negative pulse, which are then output. Furthermore, the pulser output wave PO, which is a rectangular wave, also forms a positive pulse and a negative pulse, which are then output. Note that various modifications of the pulser 110 can be executed as will be described later with reference to, for example, FIG. 7.

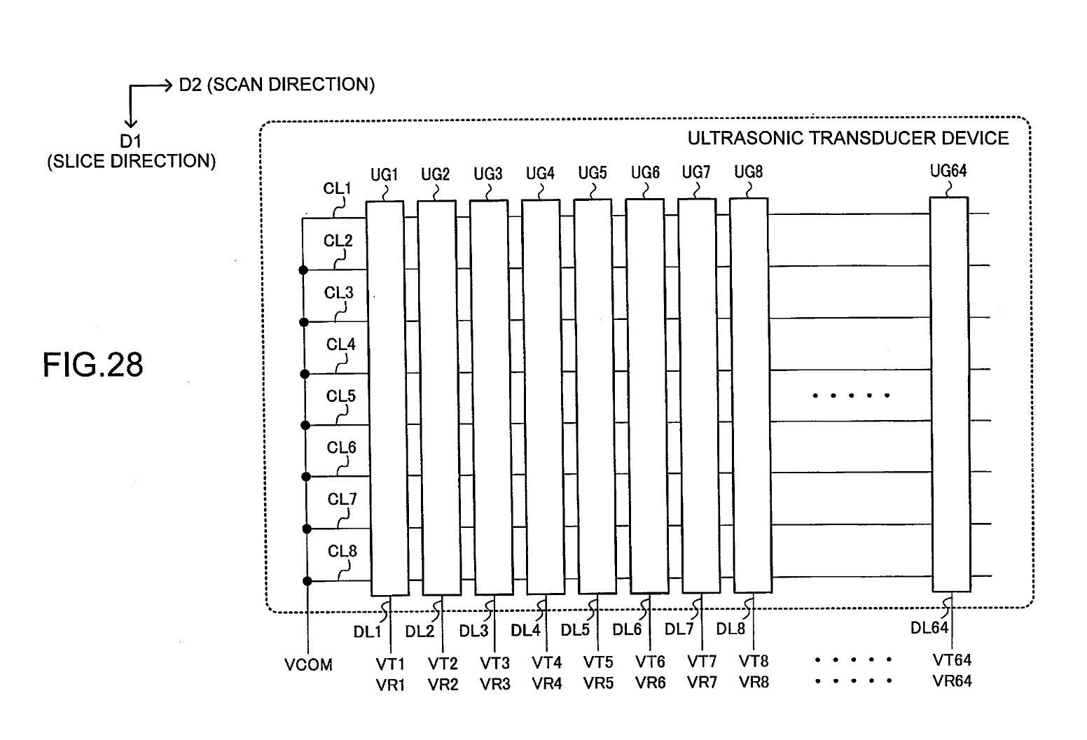

Also, the ultrasonic measurement device 100 includes the plurality of ultrasonic transducer elements that constitute the resonance circuit 120, and the plurality of ultrasonic transducer elements constitutes an ultrasonic transducer device as will be described later with reference to FIG. 28.

The ultrasonic transducer device transmits an ultrasonic beam to a subject while the subject is scanned along the scan surface, and receives an ultrasonic echo obtained by transmitting the ultrasonic beam. Taking a type using piezoelectric elements as an example, the ultrasonic transducer device includes a plurality of ultrasonic transducer elements (ultrasonic element array) and a substrate having a plurality of openings in an array. Also, ultrasonic transducer elements having a monomorphic (unimorphic) structure in which thin piezoelectric elements and a metal plate (vibrating film) are adhered to each other are used. The ultrasonic transducer elements (vibrating elements) are configured to convert electrical vibration into mechanical vibration, and are warped in this case because the size of the metal plate (vibrating film) to which they are adhered is constant even when the piezoelectric elements extend and shrink on the surface. Therefore, by applying an alternating-current voltage to the piezoelectric material film, the vibrating film vibrates in the film thickness direction, and an ultrasonic wave is emitted by the vibration of this vibrating film. Note that the voltage that is to be applied to the piezoelectric material film is, for example, 10 to 30 V, and the frequency thereof is, for example, 1 to 10 MHz.

Furthermore, the ultrasonic transducer device may have a configuration in which several ultrasonic transducer elements arranged in the neighborhood constitute one channel, and a plurality of channels are driven at once to sequentially shift the ultrasonic beam.

Note that a transducer of a type using piezoelectric elements (thin film piezoelectric elements) may be employed as the ultrasonic transducer device, but the present embodiment is not limited to this. For example, a transducer such as c-MUT (Capacitive Micro-machined Ultrasonic Transducers) of a type using capacitive elements, or a bulk-type transducer may be employed. More detailed descriptions of the ultrasonic transducer elements and the ultrasonic transducer device will be given later.

### 2.2. Detail of Processing

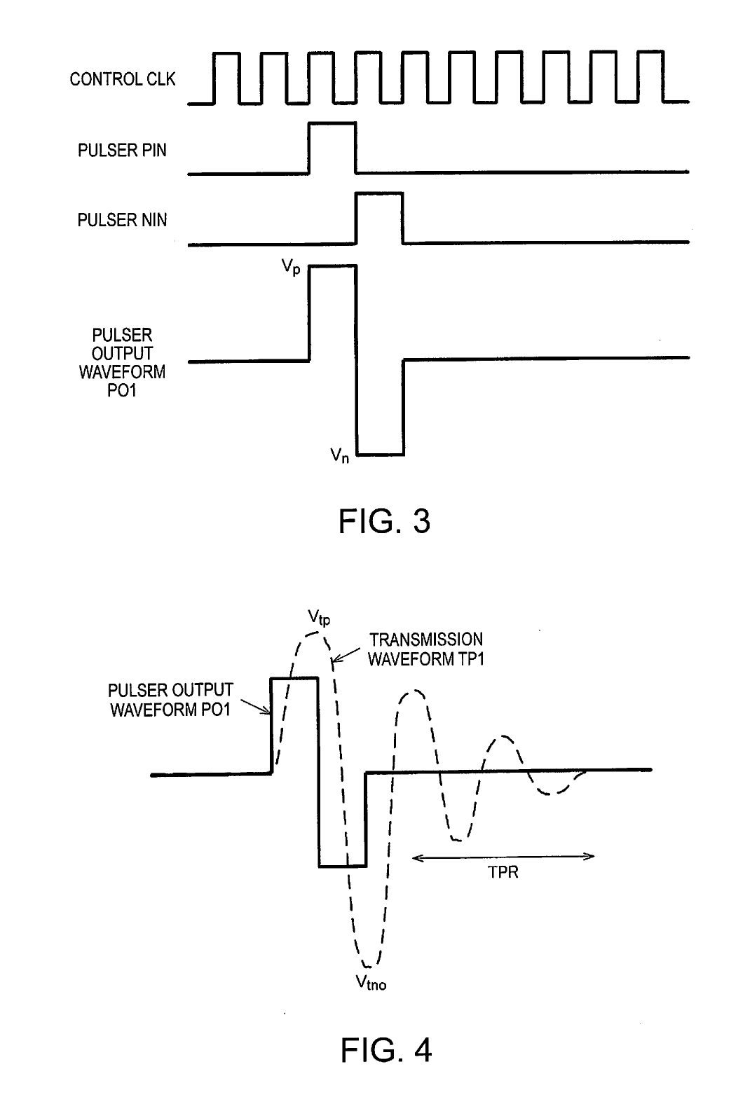

Hereinafter, processing of the present embodiment will be described in detail. First, a method for driving the pulser 110 and a pulser output wave PO1 are shown in FIG. 3, and the pulser output wave PO1 and a transmission wave TP1 that are to be input to the ultrasonic transducer elements are shown in FIG. 4 overlapping each other.

A control CLK (clock) of FIG. 3 is for use in taking timings when drive control signals PIN and NIN are generated, and is shown for use in description. The control CLK has a frequency twice as high as a frequency f₀ at which the ultrasonic transducer elements are driven, and the drive control signals are configured to be formed in synchronization with rising of the control CLK. In this example, an output waveform having one cycle of wave is formed with a combination of a positive pulse and a negative pulse by inputting the drive control signals PIN and NIN each for 1 CLK. The absolute values of the positive pulse voltage Vₚ and the negative pulse voltage Vₙ are equal to each other, that is, Vₚ=−Vₙ.

In this driving method, as shown by the transmission wave TP1 of FIG. 4, the first peak value Vₜₚ is smaller than the next peak value Vₜₙₒ. Furthermore, the transmission wave TP1 of FIG. 4 has a remaining large and long transient response TRP. These situations are caused because the driving method uses resonance characteristics of a low-pass filter and not only a drive pulse but also resonant vibration exerts.

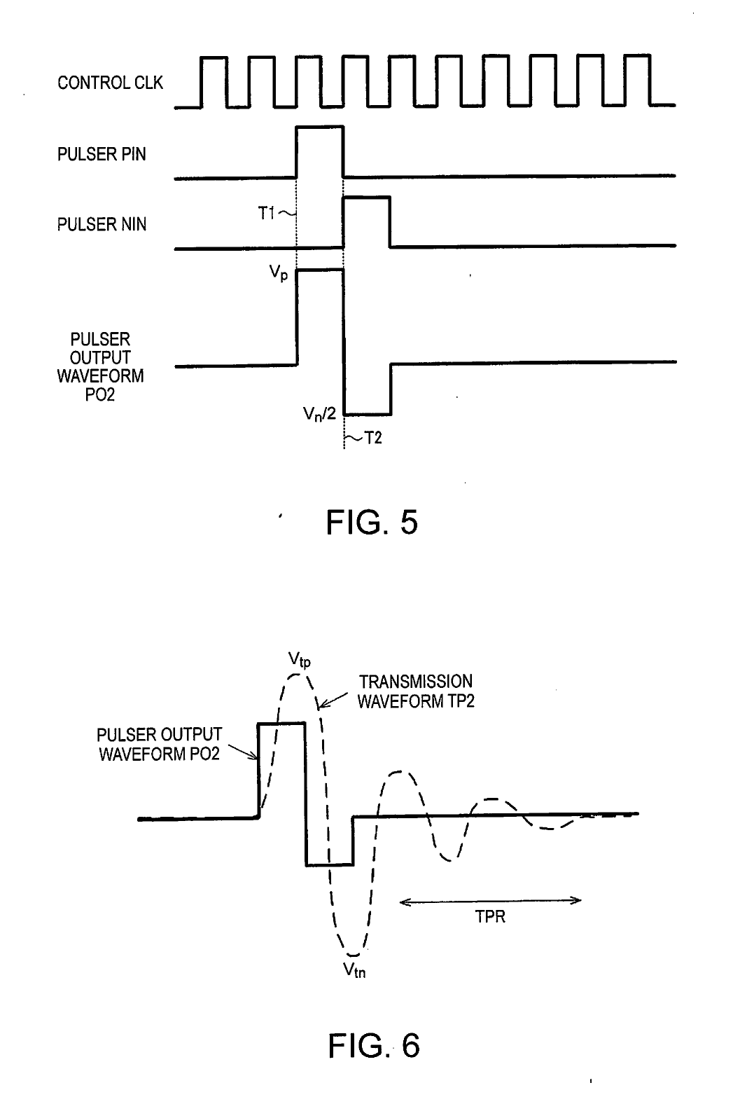

Accordingly, in the present embodiment, the following method is used to control the amplitude level or to suppress the transient response. A method for driving the pulser 110 and a pulser output wave PO2 according to a first example of the present embodiment are shown in FIG. 5, and the pulser output wave PO2 and a transmission wave TP2 that is to be input to the ultrasonic transducer elements are shown in FIG. 6 overlapping each other.

In this example, the voltage level of the negative pulse of the pulser output wave PO2 shown in FIG. 5 is set to Vₙ/2. Accordingly, the first peak value Vₜₚ and the next peak value Vₜₙ of the transmission wave TP2 shown in FIG. 6 are substantially the same. This is because by reducing the negative pulse voltage, the peak value Vₜₙₒ of the transmission wave TP1 shown in FIG. 4, which is enlarged by the negative pulse driving and resonant vibration, is suppressed.

Here, the circuit configuration of the pulser 110 according to the first example of the present embodiment is shown in FIG. 7. The pulser 110 according to the first example includes, in addition to the configuration described above with reference to FIG. 2, a switch SWₚ to which the power-supply voltage Vₚ and a power-supply voltage Vₚ/2 are input and is used for selecting any one of them, and a switch SWₙ to which the power-supply voltage Vₙ and the power-supply voltage Vₙ/2 are input and is used for selecting any one of them. Also, a pulser control signal SWP that controls the switch SWₚ is input to the switch SWₚ, and a pulser control signal SWN that controls the switch SWₙ is input to the switch SWₙ.

In the case of the example of FIG. 5, by the switch SWₙ selecting the power-supply voltage Vₙ/2 at the same timing as the drive control signal NIN based on the pulser control signal SWN, the pulser 110 outputs a pulser output wave of voltage Vₙ/2. Note that in FIG. 7, the power-supply voltage Vₚ/2 and the power-supply voltage Vₙ/2 are externally fed, but may be generated based on the power-supply voltage Vₚ and the power-supply voltage Vₙ within the pulser.

As described above, in the first example, the pulse signal output circuit 110 outputs a first pulse signal having a first pulse voltage at a first pulse output timing, and outputs a second pulse signal having a second pulse voltage, which is different from the first pulse voltage, at a second pulse output timing after the first pulse output timing.

Accordingly, it is possible to output pulse signals having different voltages at different timings, and to perform control of the amplitude of the transmission wave, suppression of the transient response, and the like.

Here, the pulse output timing is defined with reference to a rising timing of the clock signal. For example, it is possible to understand that if pulse signals are output at the same rising timings of the clock signal, the two pulse output timings are the same, and if pulse signals are output at different rising timings of the clock signal, the two pulse output timings are different. In the example of FIG. 5, the first pulse output timing is a rising timing T1 of the drive control signal PIN, and the second pulse output timing is a rising timing T2 of the drive control signal NIN, for example. The first pulse output timing T1 and the second pulse output timing T2 are different pulse output timings.

Furthermore, the first pulse signal is a first polarity pulse signal that has the positive polarity or the negative polarity, and the second pulse signal is a second polarity pulse signal that has the polarity different from the first polarity. Then, the absolute value of the second pulse voltage is smaller than the absolute value of the first pulse voltage.

In the example of FIG. 5, for example, the first pulse signal is a positive polarity pulse signal (positive pulse) that was output at the above-described first pulse output timing T1, and the second pulse signal is a negative polarity pulse signal (negative pulse) that was output at the above-described second pulse output timing T2. Note that in this case, the first polarity is the positive polarity and the second polarity is the negative polarity.

Furthermore, in the example of FIG. 5, the first pulse voltage is Vₚ, and the second pulse voltage is Vₙ/2. Furthermore, the absolute values of Vₚ and Vₙ are equal to each other, and thus the absolute value of the second pulse voltage is smaller than the absolute value of the first pulse voltage.

Accordingly, it is possible to, for example, suppress an increase in the amplitude of the transmission wave corresponding to the second pulse signal as compared with the amplitude of the transmission wave corresponding to the first pulse signal.

Furthermore, it is also possible to understand that the second pulse signal is a pulse signal for suppressing the resonant vibration of a transmission signal to the ultrasonic transducer element.

Accordingly, it is possible, for example, to suppress the resonant vibration of the transmission signal.

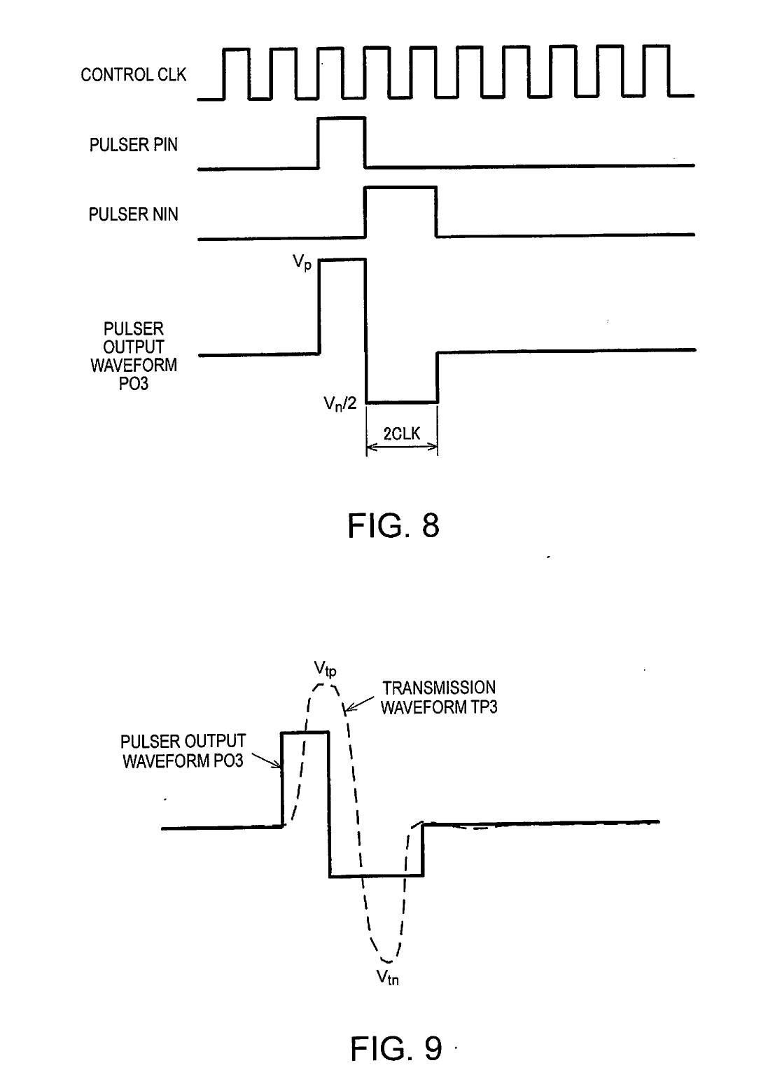

Hereinafter, a method for driving the pulser 110 and a pulser output wave PO3 according to a second example of the present embodiment are shown in FIG. 8, and the pulser output wave PO3 and a transmission wave TP3 that is to be input to the ultrasonic transducer elements are shown in FIG. 9 overlapping each other. In the first example of FIG. 5, the voltage level of the negative pulse is set to Vₙ/2, but in the second example of FIG. 8, the drive control signal NIN of the voltage Vₙ/2 is set as corresponding to 2 CLKs as well, and thereby the transient response of the transmission wave TP3 is suppressed as shown in FIG. 9. This is, the transient response that was generated by the amplitude of the transmission wave on the positive polarity side returning and increasing due to the resonant vibration is suppressed by applying a negative voltage for a longer time. In this context, the period of 2 CLKs corresponds to a driving period (1/f₀).

As described above, in the second example, the pulse signal output circuit 110 outputs the first pulse signal having a first pulse width at the first pulse output timing, and outputs the second pulse signal having a second pulse width, which is different from the first pulse width, at the second pulse output timing after the first pulse output timing. Here, the second pulse width is larger than the first pulse width. For example, in the above-described examples of FIGS. 8 and 9, the first pulse width is a 1 CLK width, and the second pulse width is a 2 CLK width.

Accordingly, it is possible to output pulse signals having different pulse widths at different timings, so as to perform control of the amplitude of the transmission wave, suppression of the transient response, and the like. For example, it is possible to perform suppression of the transient response and the like by suppressing an increase in the amplitude of the transmission wave on the positive polarity side due to resonant vibration after the amplitude of the transmission wave shows a negative value by the second pulse signal.

When the second example is described in detail in other words, the pulse signal output circuit 110 outputs the first pulse signal having the first pulse voltage and the first pulse width at a first pulse output timing, and outputs, at the second pulse output timing after the first pulse output timing, the second pulse signal having the second pulse voltage whose absolute value is smaller than that of the first pulse voltage, and the second pulse width, which is longer than that of the first pulse width.

Accordingly, it is possible, for example, to suppress an increase in the amplitude of the transmission wave corresponding to the second pulse signal as compared with that of the transmission wave corresponding to the first pulse signal, and to suppress an increase in the amplitude of the transmission wave on the positive polarity side due to resonant vibration after the amplitude of the transmission wave shows a negative value due to the second pulse signal, thereby allowing suppression of the transient response and the like.

Furthermore, it can be understood that the second pulse signal is a pulse signal for suppressing sound reverberation of the transmission signal to the ultrasonic transducer element.

Accordingly, it is possible, for example, to suppress sound reverberation (transient response) of a transmission signal.

As described above, by controlling the pulse voltage and the pulse width of rectangular wave driving, it is possible to obtain a transmission wave of a (substantial) sine wave that has a short transient response, that is, a transmission wave in which a harmonic component is removed and tailing is reduced. Accordingly, in the harmonic imaging, appropriate image generation is possible only using a harmonic component caused by a nonlinear effect because there is no reflected wave due to a harmonic component included in the transmission wave.

Furthermore, the driving method in which the low-pass filter is configured is a method using resonant vibration, and a transmission voltage that is the output voltage of the pulser 110 or greater can be obtained. Therefore, ultrasonic transducer elements that can be driven with a low voltage can be driven by an ordinary low voltage logic IC, liquid crystal display driver, or the like, and it is not necessary to use an expensive high voltage pulser IC for driving a bulk ultrasonic transducer element. Furthermore, there is an advantage that even a circuit with a large number of channels can be downsized and realized at a low cost.

The examples in which a transmission wave of one cycle is output have been described so far, but the following will further describe examples in which other numbers of transmission wave cycles are output.

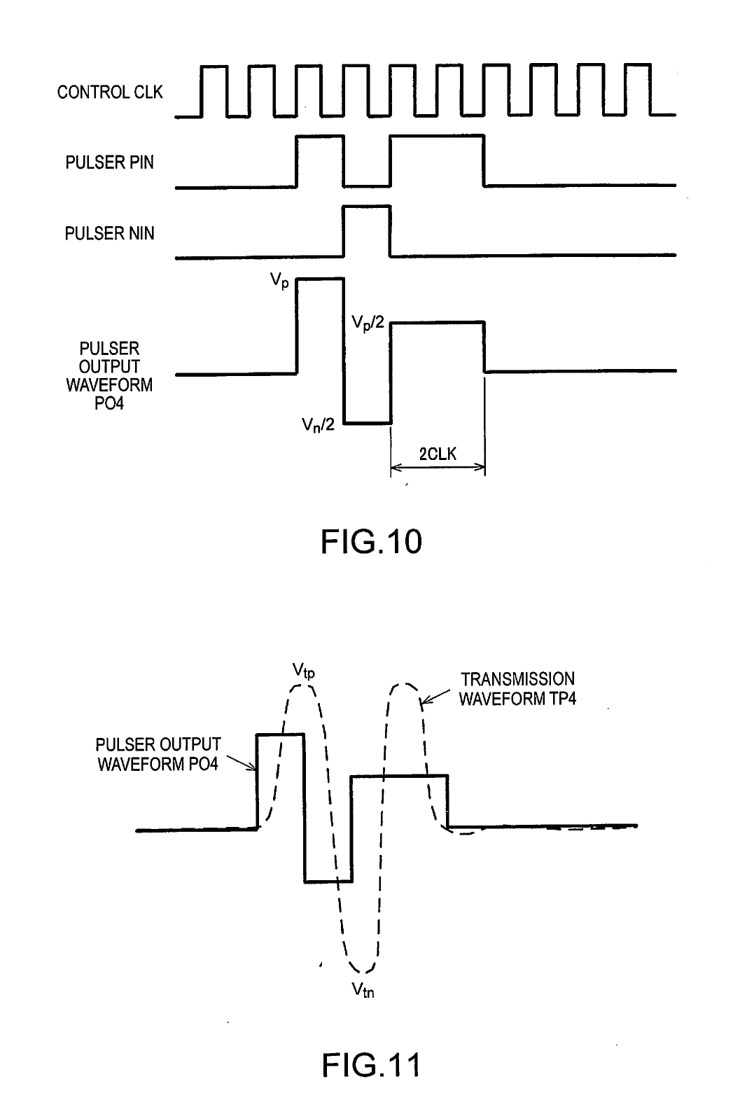

A method for driving the pulser 110 and a pulser output wave PO4 according to a third example of the present embodiment are shown in FIG. 10, and the pulser output wave PO4 and a transmission wave TP4 of one and a half cycles are shown in FIG. 11 overlapping each other. When the transmission wave of one and a half cycles is output, the pulser 110 output a positive pulse of a voltage Vₚ for 1 CLK, then outputs a negative pulse of a voltage Vₙ/2 for 1 CLK, and ultimately outputs a positive pulse of a voltage Vₚ/2 for 2 CLKs. As shown in FIG. 11, it is thus possible to obtain a transmission wave TP4 of one and a half cycles that has the shape of a (substantial) sine wave.

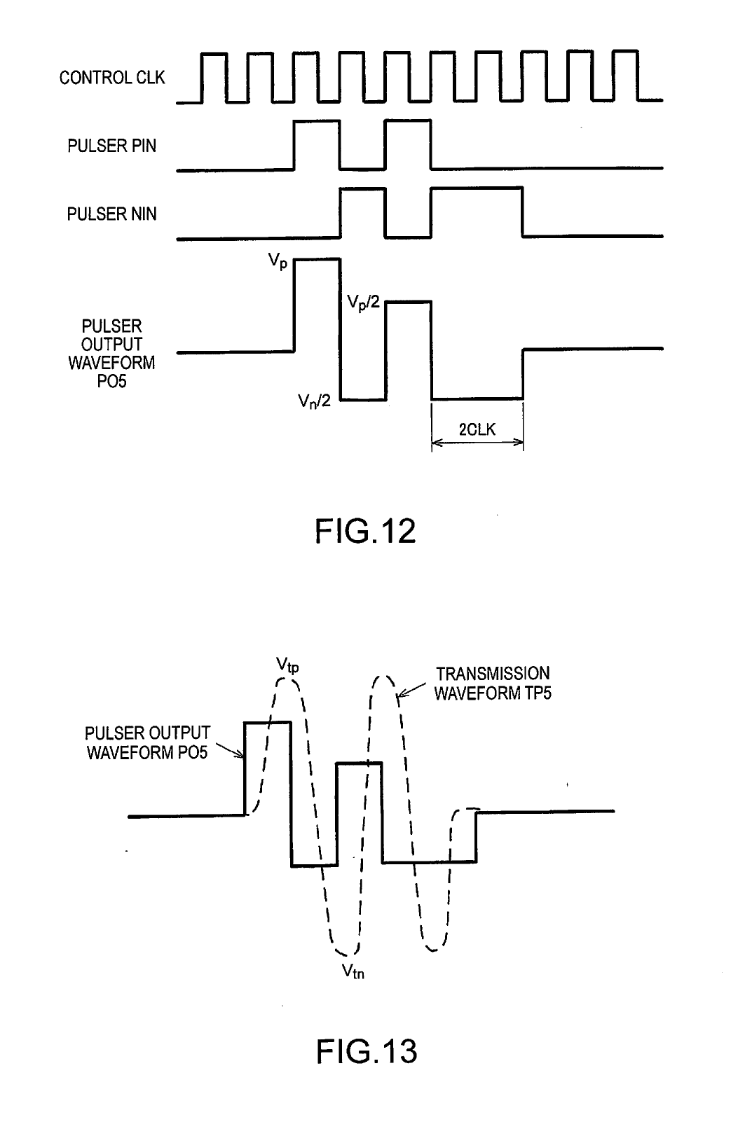

Then, a method for driving the pulser 110 and a pulser output wave PO5 according to a fourth example of the present embodiment are shown in FIG. 12, and the pulser output wave PO5 and a transmission wave TP5 of two cycles are shown in FIG. 13 overlapping each other. When a transmission wave of two cycles is output, the pulser 110 outputs a positive pulse of a voltage Vₚ for 1CLK, then outputs a negative pulse of a voltage Vₙ/2 for 1 CLK, further outputs a positive pulse of a voltage Vₚ/2 for 1 CLK, and ultimately outputs a negative pulse of a voltage Vₙ/2 for 2 CLKs. As shown in FIG. 13, it is thus possible to obtain a transmission wave TP5 of two cycles that has the shape of a (substantial) sine wave.

Similarly, a transmission wave of three cycles or more can be realized by repeating the same configuration. Furthermore, in the description above, the operation starting from a positive pulse is described, but a reversed phase driving waveform starting from a negative pulse can be formed by similar repetition of setting the voltage of the first negative pulse to Vₙ and the voltage of the next positive pulse to Vₚ/2.

This is the appropriate driving method in which an inductor L is set so that a cut-off frequency f_{c} of the low-pass filter indicated by the following formula (1) is equal to the driving frequency f₀, and a resistance R is set so that an attenuation coefficient ζ is about 0.2.

$$\begin{matrix}〈 Formula 1 〉 & \\ {f}_{c} = \frac{1}{2 π \sqrt{LC}} & ( 1 )\end{matrix}$$

In this case, L is set by the following formula (2), R of the configuration of FIG. 1A is set by the following formula (2), and R of the configuration of FIG. 1B is set by the following formula (2).

$$\begin{matrix}〈 Formula 2 〉 & \\ L = \frac{1}{4 {π}^{2} C {f}_{0}^{2}} & ( 2 ) \\ 〈 Formula 3 〉 & \\ R = 0.4 \sqrt{\frac{L}{C}} & ( 3 ) \\ 〈 Formula 4 〉 & \\ R = 2.5 \sqrt{\frac{L}{C}} & ( 4 )\end{matrix}$$

In this condition, as shown in FIG. 7, the positive polarity side power supply and the negative polarity side power supply are each configured with two stages, that is, a simple circuit configuration can be realized.

Then, a driving method in which the attenuation coefficient is smaller than that of the above-described condition and the amplitude is enlarged, and in contrast, a driving method in which the attenuation coefficient is greater than that of the above-described condition and the amplitude is suppressed will be described with reference to examples of the present embodiment.

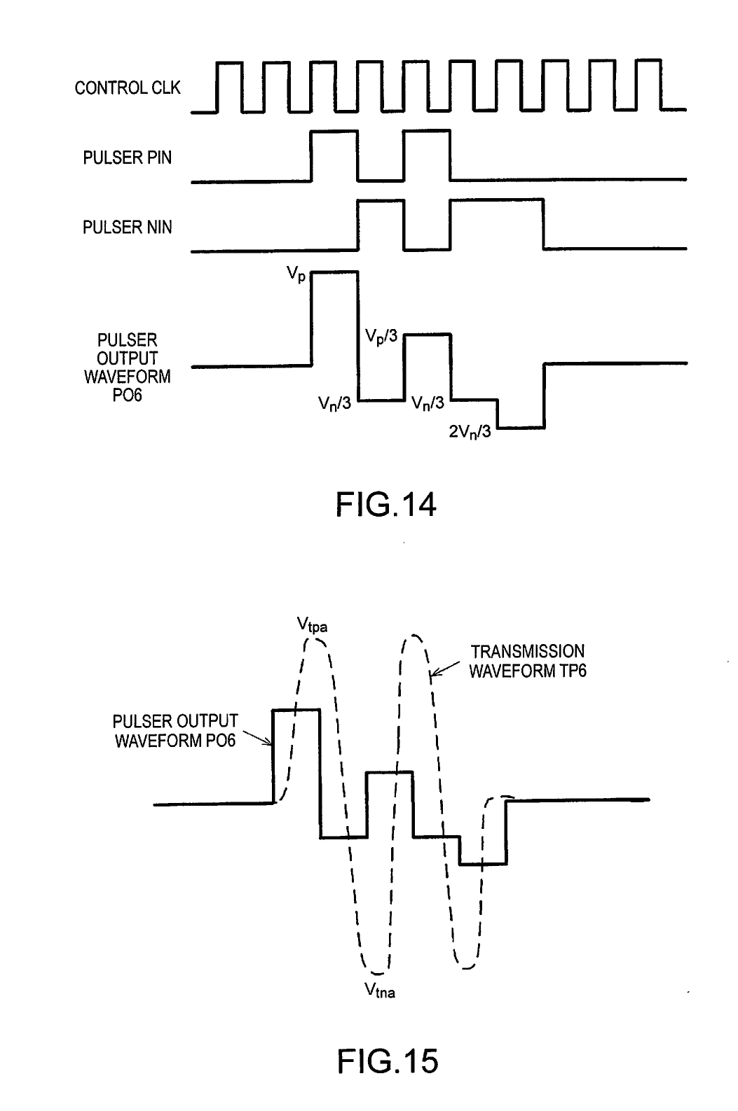

Hereinafter, a method for driving the pulser 110 and a pulser output wave PO6 according to a fifth example of the present embodiment are shown in FIG. 14, and the pulser output wave PO6 and a transmission wave TP6 of two cycles are shown in FIG. 15 overlapping each other. In this example, the attenuation coefficient is set to about 0.1.

The absolute values of peak values Vₜₚₐ and Vₜₙₐ of the transmission wave TP6 of FIG. 15 are larger than those of the peak values Vₜₚ and Vₜₙ of the transmission wave TP5 of FIG. 13, since the attenuation coefficient is reduced. In this example, since the resonance amplitude is large, the pulse voltages from the second pulse onwards are set, as shown in FIG. 14, to Vₚ/3 and Vₙ/3, whose absolute values are smaller than those of Vₚ/2 and Vₙ/2 of the fourth example of FIG. 12, in order that the positive and negative transmission amplitudes are equal to each other. Furthermore, the last pulse voltage is set to 2Vₙ/3, whose absolute value is larger than Vₙ/3, in order to suppress the transient response having a larger amplitude. Accordingly, even when the attenuation coefficient is set to a small value, it is possible to obtain a transmission wave of two cycles that has the shape of a (substantial) sine wave, achieving a suppressed transient response.

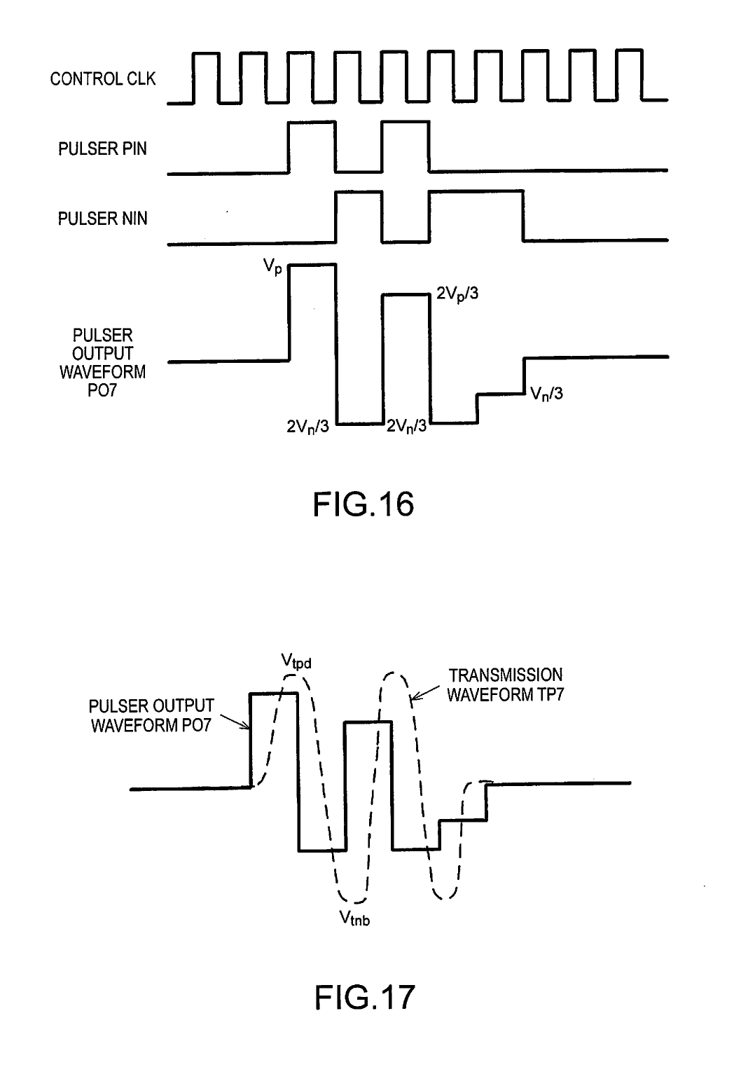

Then, a method for driving the pulser 110 and a pulser output wave PO7 according to a sixth example of the present embodiment are shown in FIG. 16, and the pulser output wave PO7 and a transmission wave TP7 of two cycles are shown in FIG. 17 overlapping each other. In this example, the attenuation coefficient is set to about 0.3.

The absolute values of peak values V_{tpd} and V_{tnb} of the transmission wave TP7 of FIG. 17 are larger than those of the peak values Vₜₚ and Vₜₙ of the transmission wave TP5 of FIG. 13, since the attenuation coefficient is increased. In this example, since the resonance amplitude is small, the pulse voltages of the second pulse onwards are set, as shown in FIG. 16, to 2Vₚ/3 and 2Vₙ/3, whose absolute values are larger than those of Vₚ/2 and Vₙ/2 of FIG. 12, in order that the positive and negative transmission amplitudes are equal to each other. Furthermore, since the amplitude of the transient response is small, the last pulse voltage is set to a small voltage Vₙ/3. Accordingly, even when the attenuation coefficient is set to a large value, it is possible to obtain a transmission wave of two cycles that has the shape of a (substantial) sine wave, achieving a suppressed transient response.

As described above, by optimizing the values of the pulse voltages of the second pulse onwards and the pulse voltage that is ultimately applied, it is possible to obtain a transmission wave of a (substantial) sine wave that has a suppressed transient response, according to the attenuation coefficient of the configured low-pass filter. Note that all the configurations of the pulser 110 in these cases include three stages on each of the positive and negative power supplies, although they are not shown.

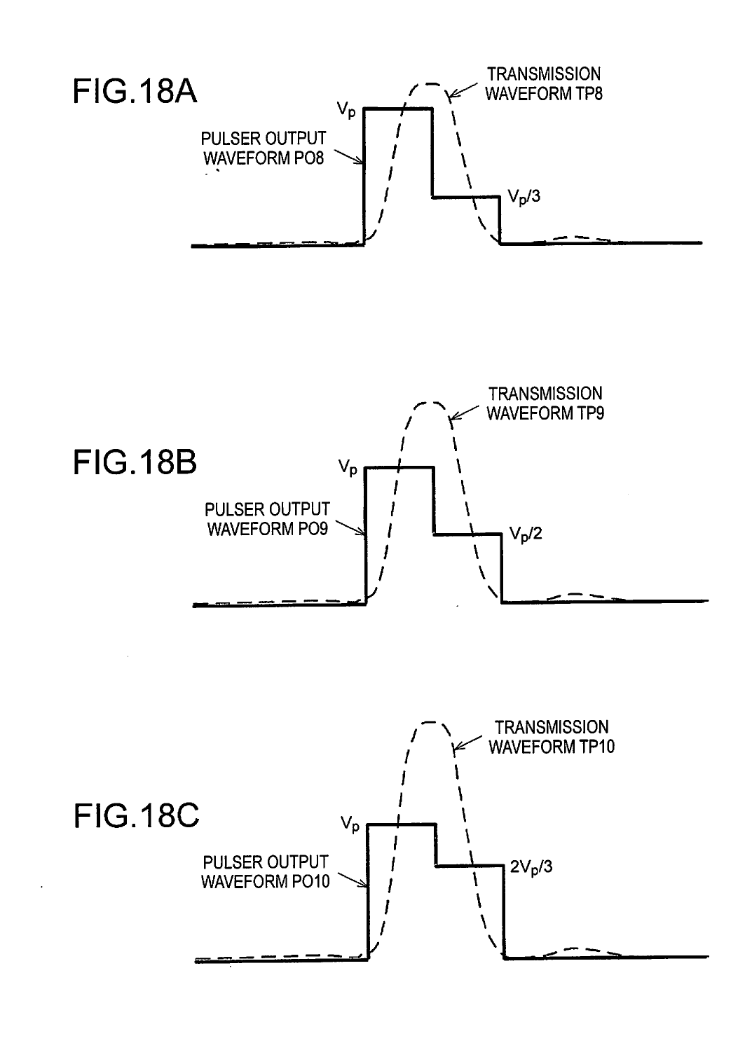

The following will describe a seventh example in which a transmission wave of a half cycle (0.5 cycle) is output. FIGS. 18A to 18C are diagrams in which pulser output waveforms (PO8 to PO10) and transmission waveforms (TP8 to TP10) that are to be input to the ultrasonic transducer elements according to the present embodiment are shown overlapping each other. FIG. 18A shows a case where the attenuation coefficient is about 0.3, FIG. 18B shows a case where the attenuation coefficient is about 0.2, and FIG. 18C shows a case where the attenuation coefficient is about 0.1.

In the case of a wave of a half cycle, a pulse for suppressing the transient response is added, and in FIG. 18A, it is the reverse voltage (Vₚ/3) that corresponds to the last pulse of FIG. 16, in FIG. 18B, it is the reverse voltage (Vₚ/2) that corresponds to the last pulse of FIG. 12, and in FIG. 18C, it is the reverse voltage (2Vₚ/3) that corresponds to the last pulse of FIG. 14. Accordingly, by optimizing the values of the pulse voltages that are ultimately applied, it is possible to obtain transmission waves of a half cycle that has the shape of a (substantial) sine wave and a suppressed transient response, according to the attenuation coefficient of the configured low-pass filter.

### 3. Second Embodiment

In the above-described first embodiment, the transmission waveforms themselves are approximates to a sine wave curve, but in the present embodiment, by approximating the envelope curve of a transmission waveform to a sine wave curve, a harmonic component of the transmission wave is suppressed.

Conventionally, a method for generating a transmission waveform using a similar approach has been proposed, but in the conventional method, there are the problems that a control method is difficult, a plurality of voltage supplies are needed, and the like.

Therefore, in the present embodiment, the envelope curve of a transmission waveform is approximated to a (substantial) sine wave curve using a simple timing control of rectangular wave driving, without increasing the number of voltage supplies as compared to a predetermined number. Accordingly, the transient response of the transmission wave is shortened.

An example of a configuration of a system of the present embodiment is the same as the configuration described above with reference to FIGS. 1A and 1B. Furthermore, the configuration of the pulser 110 is the same as the configuration described above with reference to FIGS. 2, 7, and the like.

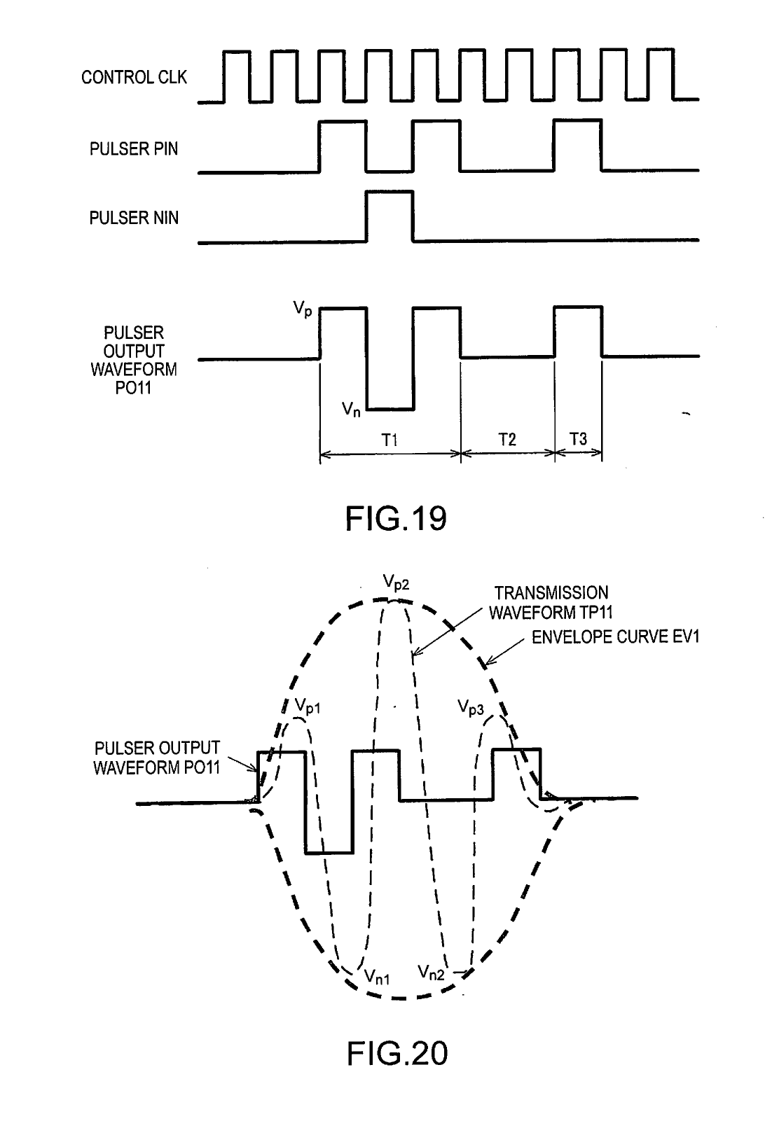

Hereinafter, processing of the present embodiment will be described in detail. First, a method for driving the pulser 110 and a pulser output wave PO11 according to the first example are shown in FIG. 19, and the pulser output wave PO11 and a transmission wave TP11 that is to be input to the ultrasonic transducer elements are shown in FIG. 20 overlapping each other.

In the present example, as shown in FIG. 20, a case where an envelope curve EV1 of the transmission waveform TP11 of two and a half cycles is a (substantial) sine wave curve will be described. Note that the inductor L of the FIG. 1A or 1B is set so that the cut-off frequency f_{c} of the low-pass filter indicated by the above formula (1) is equal to the driving frequency f₀.

In the present example, by applying in sequence pulse signals whose voltages are Vₚ−Vₙ−Vₚ in the stated order as shown in FIG. 19, the peak values of the transmission wave TP11 are set to Vₚ₁−Vₙ₁−Vₚ₂ in the stated order due to a resonance effect (see FIG. 20). Because there is thereafter a time period in which no pulse voltage is applied, the peak values of the transmission wave TP11 are reduced due to resonance attenuation so as to be Vₙ₂−Vₚ₃ in the stated order. At that time, the resonance attenuation coefficient is optimized by setting the resistance R of FIG. 1A or 1B so that Vₚ₁≈Vₚ₃ and Vₙ₁≈Vₙ₂ are satisfied. Ordinarily, the resistance R with respect to a desired attenuation coefficient ζ is indicated by the below formula (5) for FIG. 1A and the below formula (6) for FIG. 2B.

$$\begin{matrix}〈 Formula 5 〉 & \\ R = 2 ζ \sqrt{\frac{L}{C}} & ( 5 ) \\ 〈 Formula 6 〉 & \\ R = \frac{1}{2 ζ \sqrt{\frac{C}{L}}} & ( 6 )\end{matrix}$$

If this goes on, vibration due to resonance sound reverberation will remain ultimately, and thus a positive pulse in the direction in which the vibration is suppressed is ultimately applied, suppressing the transient response at minimum.

In summary of the above-described first example, the pulse signal output circuit 110 outputs one or more first time period pulse signals during a first time period, outputs no pulse signal during a second time period after the first time period, and outputs a third time period pulse signal during a third time period after the second time period.

In the example of FIG. 19, for example, the first time period is the time period denoted by T1 and the sequential pulse signals whose voltages are Vₚ−Vₙ−Vₚ are the plurality of first time period pulse signals. Also, the second time period is the time period denoted by T2, and no pulse signal is output in the second time period.

Also, the third time period is the time period denoted by T3, and the pulse signal output circuit 110 outputs, during the third time period, a pulse signal for suppressing sound reverberation of the transmission signal to the ultrasonic transducer elements.

Accordingly, suppression of sound reverberation (transient response) of a transmission signal and the like are possible.

Similarly to the pulse output timing, each of the first to third time periods is defined based on a rising timing of a clock signal. Each of the first to third time period is a time period between a first rising timing and a second rising timing after the first rising timing of the clock signal. The length of the time period is arbitrary.

As described above, in the present embodiment, it is possible to approximate the envelope curve of a transmission waveform to a (substantial) sine wave curve using simple timing control of rectangular wave driving, without increasing the number of voltage supplies, thereby shortening the transient response of the transmission wave.

In other words, the pulse signal output circuit 110 outputs a pulse signal that approximates the envelope curve of the waveform of a transmission signal to the ultrasonic transducer elements to a sine wave shape.

Accordingly, when performing, for example, harmonic imaging, appropriate image generation and the like are possible only using a harmonic component caused by a nonlinear effect because there is no reflected wave due to a harmonic component included in the transmission wave.

Also, in the present embodiment, a driving method in which a low-pass filter is configured is a method using resonant vibration, and a transmission voltage that is the output voltage of the pulser 110 or more can be obtained. Therefore, ultrasonic transducer elements that can be driven with a low voltage can be driven by an ordinary low voltage logic IC, liquid crystal display driver, or the like, and it is not necessary to use an expensive high voltage pulser IC for driving a bulk ultrasonic transducer element, realizing an effect that even a circuit with a large number of channels can be downsized and realized at a low cost.

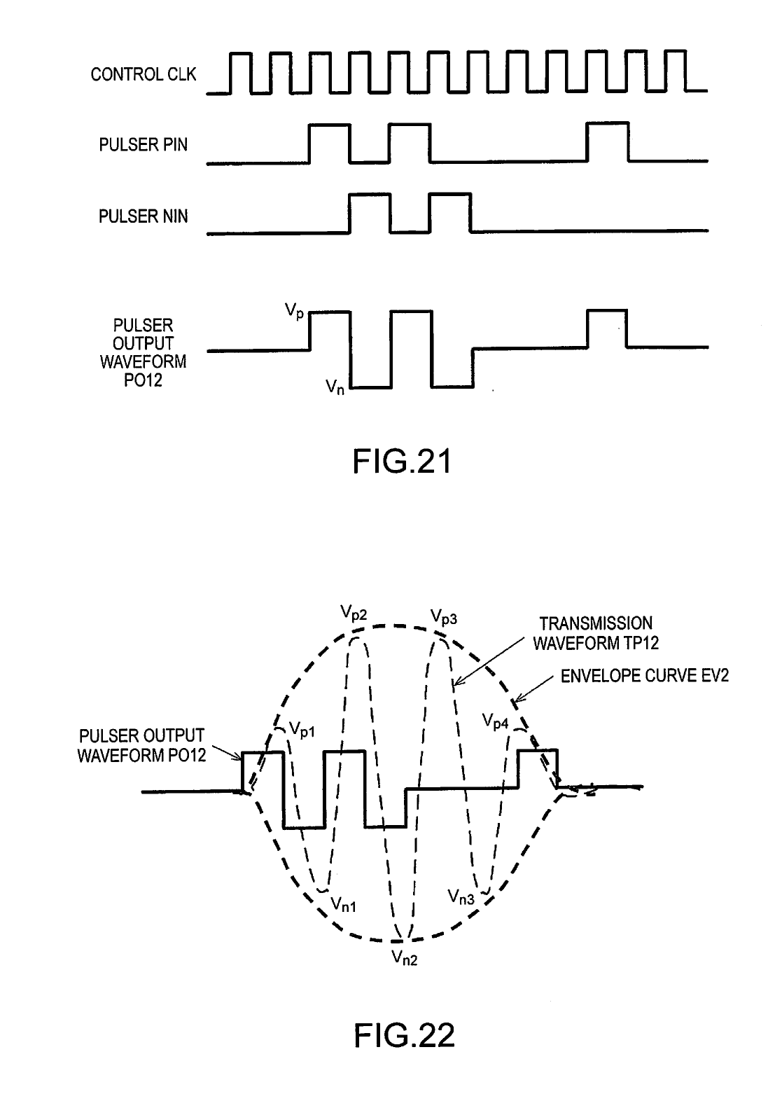

Hereinafter, a method for driving the pulser 110 and a pulser output wave PO12 according to a second example are shown in FIG. 21, and the pulser output wave PO12 and a transmission wave TP12 that is to be input to the ultrasonic transducer elements are shown in FIG. 22 overlapping each other.

The present example describes a case where, as shown in FIG. 22, an envelope curve EV2 of the transmission waveform TP12 of three and a half cycles is a (substantial) sine wave curve. Note that similarly to the above-described first example, the inductor L of the FIG. 1A or 1B is set so that the cut-off frequency f_{c} of the low-pass filter is equal to the driving frequency f₀.

In the present example, by applying in sequence pulse signals whose voltages are Vₚ−V_{c}−Vₚ−Vₙ in the stated order as shown in FIG. 21, the peak values of the transmission wave TP12 are set to Vₚ₁−Vₙ₁−Vₚ₂−Vₙ₂ in the stated order due to a resonance effect (see FIG. 22). Because there is thereafter a time period in which no pulse voltage is applied, the peak values of the transmission wave TP12 is reduced due to resonance attenuation, and are Vₚ₃−Vₙ₃−Vₚ₄ in the stated order. At that time, the resonance attenuation coefficient is optimized by setting the resistance R of FIG. 1A or 1B so that Vₚ₁≈Vₚ₄, Vₙ₁≈Vₙ₃, and Vₚ₂≈Vₚ₃ are satisfied.

Furthermore, if this goes on, vibration due to resonance sound reverberation will remain ultimately, and thus a positive pulse of the voltage Vₚ in the direction in which the vibration is suppressed is ultimately applied, suppressing the transient response to moderate dumping at minimum. Accordingly, even when the number of cycles of wave is increased, the same effect as that of the first example can be achieved.

The above-described examples are examples in which the number of cycles of transmission wave is two and a half and three and a half, but the following will describe an example in which the number of cycles of wave is an integer, such as a case of two cycles or three cycles.

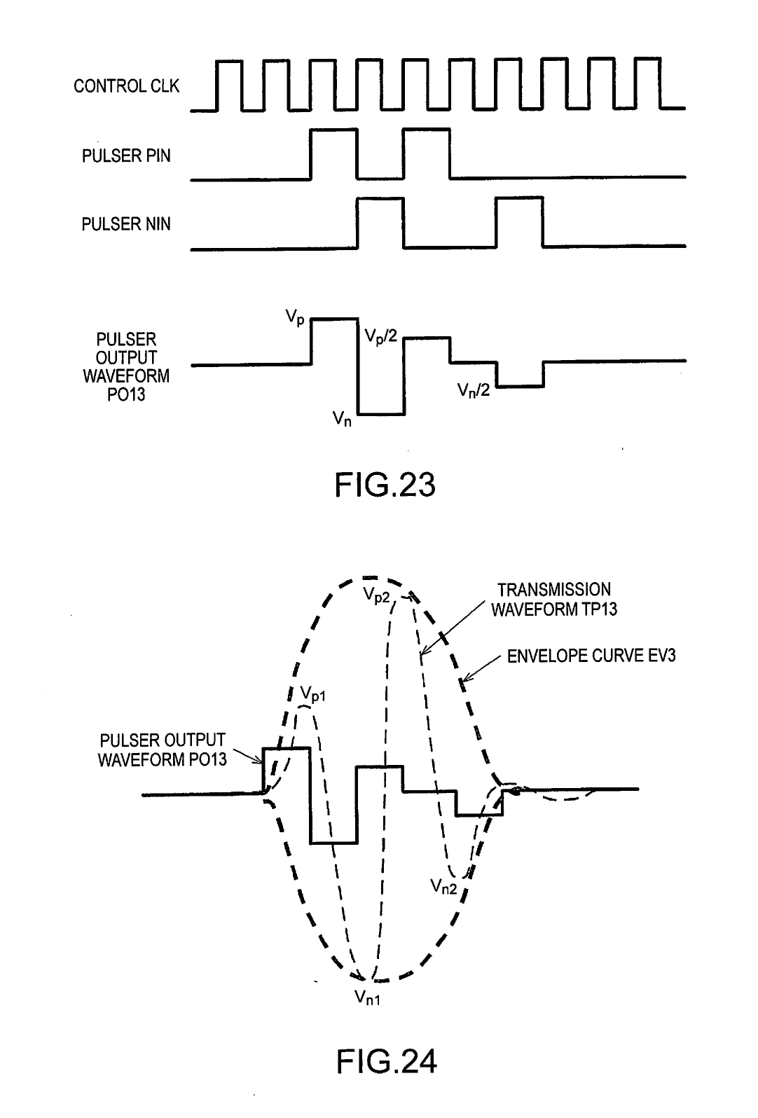

Hereinafter, a method for driving the pulser 110 and a pulser output wave PO13 according to a third example is shown in FIG. 23, and the pulser output wave PO13 and a transmission wave TP13 that is to be input to the ultrasonic transducer elements are shown in FIG. 24 overlapping each other.

The present example will describe a case where an envelope curve EV3 of the transmission waveform TP13 of two cycles is a (substantial) sine wave curve, as shown in FIG. 24. Note that the inductor L of FIG. 1A or 1B is set so that the cut-off frequency f_{c} of the low-pass filter indicated by the above-described formula (1) is equal to the driving frequency f₀. Furthermore, the third example uses the pulser 110 having the configuration shown in FIG. 7.

In the present example, by applying in sequence pulse signals whose voltages are Vₚ−Vₙ in the stated order as shown in FIG. 23, the peak values of the transmission wave TP13 are set to Vₚ₁−Vₙ₁ in the stated order due to a resonance effect (see FIG. 24). Furthermore, a positive pulse of the voltage Vₚ/2 is continuously applied in order to satisfy Vₚ₂≈−Vₙ₁. After a time period in which no pulse voltage is applied, a negative pulse Vₙ/2 in the direction in which the vibration due to resonance sound reverberation is suppressed is applied ultimately. Accordingly, the peak values of the transmission wave TP11 are reduced due to resonance attenuation so as to be Vₚ₂−Vₙ₂ in the stated order. At that time, the resonance attenuation coefficient is optimized by setting the resistance R of FIG. 1A or 1B so that Vₚ/2=−Vₙ/2, Vₚ₂≈−Vₙ₁, and Vₚ₁≈−Vₙ₂ are satisfied.

In this case, it is possible to obtain a transmission wave in which the envelope curve EV3 of the transmission waveform is a (substantial) sine wave curve, only by incrementing the number of the voltage supplies of the pulser 110 by 1 with respect to that of the pulser 110 having the configuration of FIG. 2. Accordingly, the same effect as that of the first example can be achieved.

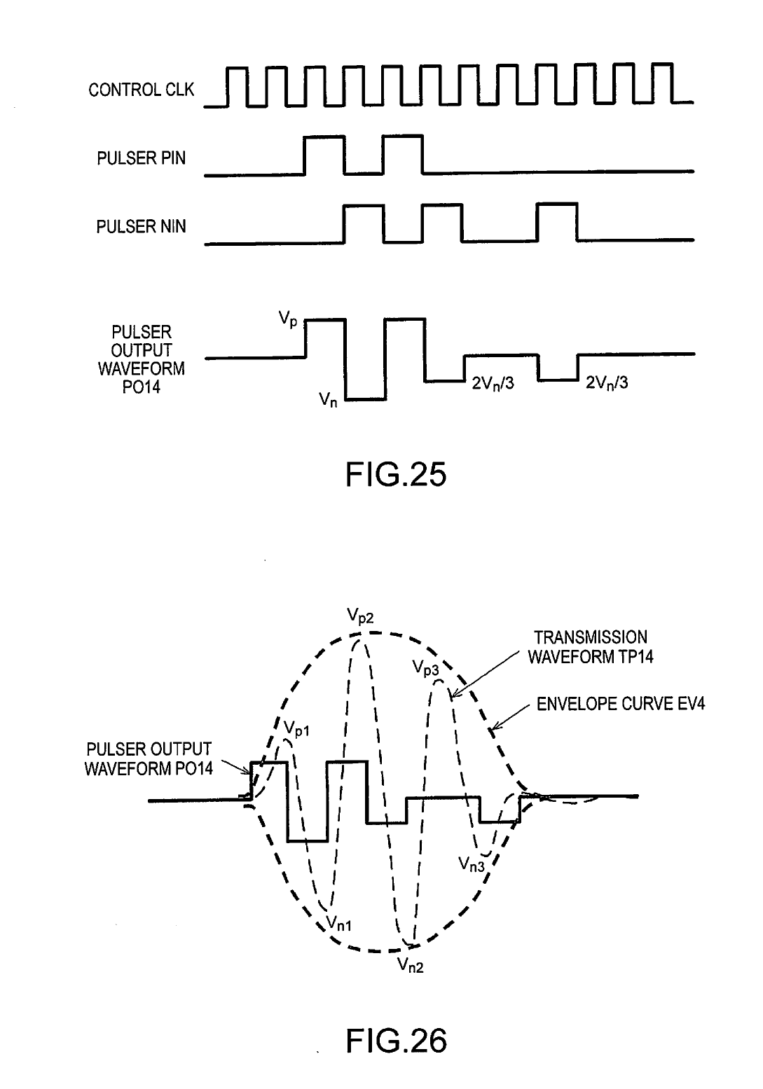

Hereinafter, a method for driving the pulser 110 and a pulser output wave PO14 according to a fourth example are shown in FIG. 25, and the pulser output wave PO14 and a transmission wave TP14 that is to be input to the ultrasonic transducer elements are shown in FIG. 26 overlapping each other. The present example will describe a case where, as shown in FIG. 26, an envelope curve EV4 of the transmission waveform TP14 of three cycles is a (substantial) sine wave curve.

In the present example, by applying in sequence pulse signals whose voltages are Vₚ−Vₙ−Vₚ in the stated order as shown in FIG. 25, the peak values of the transmission wave TP14 are set to Vₚ₁−Vₙ₁−Vₚ₂ in the stated order due to a resonance effect (see FIG. 26). Furthermore, a negative pulse of the voltage 2Vₙ/3 is continuously applied in order to satisfy Vₚ₂≈−Vₙ₂. After a time period in which no pulse voltage is applied, a negative pulse 2Vₙ/3 in the direction in which the vibration due to resonance sound reverberation is suppressed is applied ultimately. Accordingly, the peak values of the transmission wave TP14 are reduced due to resonance attenuation so as to be Vₚ₃−Vₙ₃ in the stated order. At that time, a resonance attenuation coefficient is optimized by setting the resistance R of FIG. 1A or 1B so that 2Vₚ/3=−2Vₙ/3, Vₚ₂≈−Vₙ₂, Vₚ₁≈−Vₙ₃, and Vₚ₃≈−Vₙ₁ are satisfied.

In this case, it is possible to obtain a transmission wave in which the envelope curve EV4 of the transmission waveform is a (substantial) sine wave curve, only by incrementing the number of the voltage supplies of the pulser 110 by at least 1 with respect to that of the pulser 110 having the configuration of FIG. 2 although the value of the voltage that is to be applied to the pulser 110 is different from that of the third example. Accordingly, the same effect as that of the first example can be achieved.

### 4. Ultrasonic Transducer Element

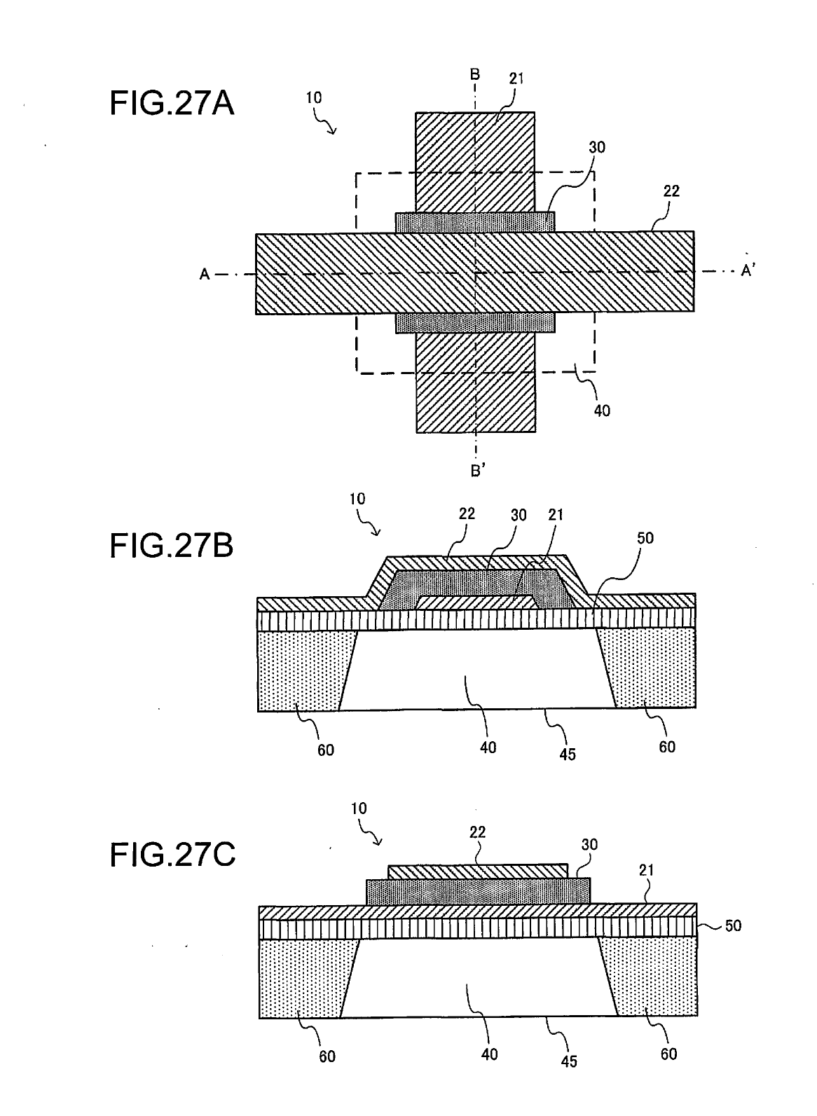

FIGS. 27A to 27C show an example of a configuration of an ultrasonic transducer element 10 of the ultrasonic transducer device. This ultrasonic transducer element 10 includes a vibrating film (membrane, supporting member) 50 and a piezoelectric element section. The piezoelectric element section includes a first electrode layer (lower electrode) 21, a piezoelectric material layer (piezoelectric material film) 30, and a second electrode layer (upper electrode) 22.

FIG. 27A is a plan view of the ultrasonic transducer element 10 that is formed on a substrate (silicon substrate) 60, viewed in the direction perpendicular to the substrate 60 on the element forming surface side. FIG. 27B is a cross-sectional view taken along the line A-A′ of FIG. 27A. FIG. 27C is a cross-sectional view taken along the line B-B′ of FIG. 27A.

The first electrode layer 21 is made from, for example, a metal thin film, and is formed on the upper layer of the vibrating film 50. This first electrode layer 21 may extend to the outside of an element forming region as shown in FIG. 27A, and may be an interconnect connected to an adjacent ultrasonic transducer element 10.

The piezoelectric material layer 30 is made from, for example, a PZT (zirconate titanate) thin film, and is provided so as to cover at least a part of the first electrode layer 21. Note that the material of the piezoelectric material layer 30 is not limited to PZT and may be made from, for example, lead titanate (PbTiO₃), lead zirconate (PbZrO₃), lead lanthanum titanate ((Pb, La)TiO₃), or the like.

The second electrode layer 22 is made from, for example, a metal thin film, and is provided so as to cover at least a part of the piezoelectric material layer 30. This second electrode layer 22 extends to the outside of the element forming region as shown in FIG. 27A, and may be an interconnect connected to an adjacent ultrasonic transducer element 10.

The vibrating film (membrane) 50 has a two-layer structure of, for example, a SiO₂ thin film and a ZrO₂ thin film, and is provided so as to cover the opening 40. This vibrating film 50 supports the piezoelectric material layer 30 and the first and second electrode layers 21 and 22, and vibrates in accordance with the expansion and contraction of the piezoelectric material layer 30, so as to be able to generate an ultrasonic wave.

The opening 40 is formed by performing etching such as reactive ion etching (RIE) on the rear surface (on which no element is formed) of the substrate 60 (silicon substrate). The resonance frequency of the ultrasonic wave is determined depending on the size of an open section 45 of the opening 40, and the ultrasonic wave is emitted to the piezoelectric material layer 30 side (in the direction from back to front of FIG. 27A).

The lower electrode (first electrode) of the ultrasonic transducer element 10 is formed by the first electrode layer 21, and the upper electrode (second electrode) thereof is formed by the second electrode layer 22. Specifically, the section of the first electrode layer 21 that is covered with the piezoelectric material layer 30 forms the lower electrode, and the section of the second electrode layer 22 that covers the piezoelectric material layer 30 forms the upper electrode. That is, the piezoelectric material layer 30 is provided between the lower electrode and the upper electrode.

### 5. Ultrasonic Transducer Device

FIG. 28 shows an example of a configuration of an ultrasonic transducer device (component chip). The ultrasonic transducer device according to the present configuration example includes a plurality of ultrasonic transducer element groups UG1 to UG64, drive electrode lines DL1 to DL64 (in a broad sense, first to n-th drive electrode lines, where n is an integer of 2 or greater), and common electrode lines CL1 to CL8 (in a broad sense, first to m-th common electrode line, where m is an integer of 2 or greater). Note that the number (n) of the drive electrode lines or the number (m) of the common electrode line are not limited to the numbers shown in FIG. 28.

The plurality of ultrasonic transducer element groups UG1 to UG64 are arranged in sixty-four lines in a second direction D2 (scan direction). Each of the ultrasonic transducer element groups UG1 to UG64 has a plurality of ultrasonic transducer elements that are arranged in a first direction D1 (slice direction).

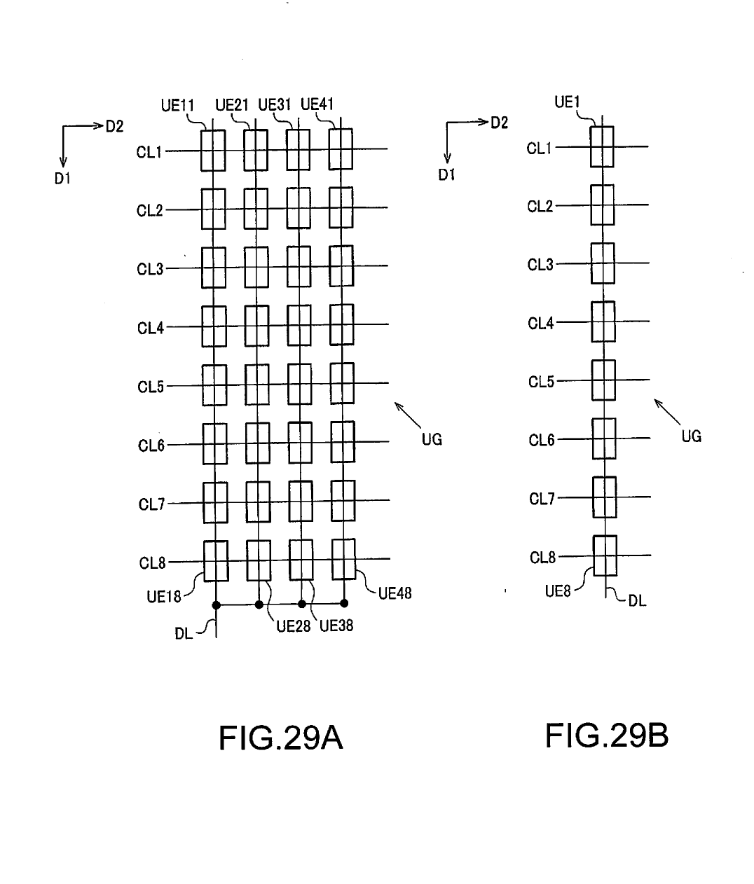

FIG. 29A shows an example of an ultrasonic transducer element group UG (one of UG1 to UG64). In FIG. 29A, an ultrasonic transducer element group UG is constituted by the first to fourth element lines. The first element line is constituted by ultrasonic transducer elements UE11 to UE18 that are arranged in the first direction D1, and the second element line is constituted by ultrasonic transducer elements UE21 to UE28 that are arranged in the first direction D1. The same applies to the third element line (UE31 to UE38) and the fourth element line (UE41 to UE48). A drive electrode line DL (one of DL1 to DL64) is connected in common to the first to fourth element lines. Furthermore, the common electrode lines CL1 to CL8 are connected to the ultrasonic transducer elements of the first to fourth element lines.

Also, the ultrasonic transducer element group UG of FIG. 29A constitute one channel of the ultrasonic transducer device. That is, the drive electrode line DL corresponds to a drive electrode line of one channel, and a transmission signal for one channel from the transmission circuit is input to the drive electrode line DL. Furthermore, a reception signal for one channel from the drive electrode line DL is output from the drive electrode line DL. Note that the number of element lines constituting one channel is not limited to four as shown in FIG. 29A, and may be less or more than four. For example, as shown in FIG. 29B, one element line may constitute one channel.

As shown in FIG. 28, the drive electrode lines DL1 to DL64 (first to n-th drive electrode lines) are arranged in the first direction D1. The j-th (where j is an integer of 1≦j≦n) drive electrode line DLj (j-th channel) among the drive electrode lines DL1 to DL64 is connected to the first electrode (for example, the lower electrode) of an ultrasonic transducer element of the j-th ultrasonic transducer element group UGj.

During a transmission time period in which an ultrasonic wave is emitted, transmission signals VT1 to VT64 are supplied to the ultrasonic transducer elements via the drive electrode lines DL1 to DL64. Furthermore, during a reception time period in which ultrasonic echo signals are received, reception signals VR1 to VR64 from the ultrasonic transducer elements are output via the drive electrode lines DL1 to DL64.

The common electrode lines CL1 to CL8 (first to m-th common electrode lines) are arranged in the second direction D2. The second electrodes of the ultrasonic transducer elements are each connected to the corresponding one of the common electrode lines CL1 to CL8. Specifically, as shown in FIG. 28 for example, the i-th (where i is an integer of 1≦i≦m) common electrode line CLi among the common electrode lines CL1 to CL8 is connected to the second electrodes (for example, the upper electrodes) of the ultrasonic transducer elements arranged in the i-th row. A common voltage VCOM is supplied to the common electrode lines CL1 to CL8. This common voltage VCOM only needs to be a constant direct-current voltage, and is not necessarily 0V, namely, a ground electric potential (ground potential). However, the present embodiment is not limited to this, and, for example, common electrode lines that are put together for ultrasonic transducer elements may respectively be drawn from the ultrasonic transducer elements, and may directly be connected to the common voltage VCOM.

During the transmission time period, a voltage corresponding to a difference between a transmission signal voltage and a common voltage is applied to the ultrasonic transducer elements, and an ultrasonic wave with a predetermined frequency is emitted.

Note that the arrangement of the ultrasonic transducer elements is not limited to the matrix arrangement shown in FIG. 28, and may be a so-called staggered arrangement or the like.

Furthermore, FIGS. 29A and 29B illustrate a case where one ultrasonic transducer element is used as both a transmission element and a reception element, but the present embodiment is not limited to the case. For example, ultrasonic transducer elements for transmission elements and ultrasonic transducer elements for reception elements are provided in a separate manner, and may be arranged in an array.

### 6. Ultrasonic Imaging Device

The ultrasonic imaging device according to the present embodiment includes the above-described ultrasonic measurement device 100 and a display unit 300 that displays image data for display that is generated based on an ultrasonic echo in response to a transmitted ultrasonic wave. The display unit 300 can be realized by, for example, a liquid crystal display, an organic EL display, an electric paper, or the like.

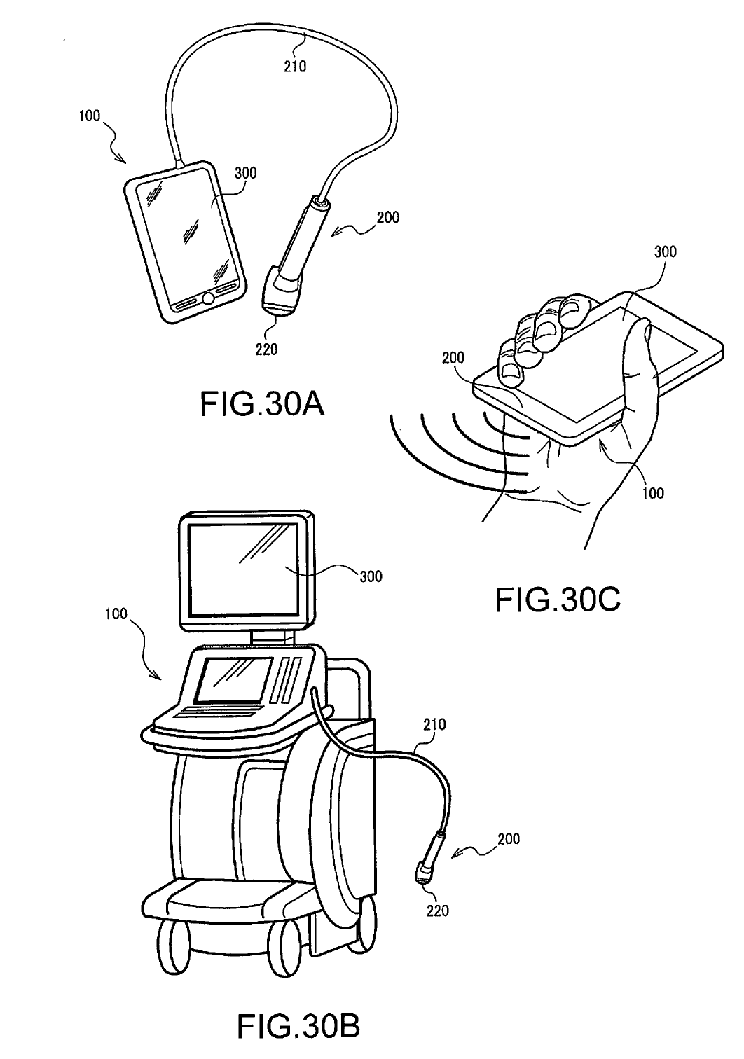

Here, examples of specific device configurations of the ultrasonic imaging device (in a broad sense, electronic device) according to the present embodiment are shown in FIGS. 30A to 30C. FIG. 30A illustrates an example of a handy-type ultrasonic imaging device, and FIG. 30B illustrates an example of a stationary-type ultrasonic imaging device. FIG. 30C illustrates an example of an integral-type ultrasonic imaging device that includes, in its main body, an ultrasonic probe 200.

The ultrasonic imaging devices of FIGS. 30A and 30B include an ultrasonic probe 200 and an ultrasonic measurement device 100, the ultrasonic probe 200 and the ultrasonic measurement device 100 being connected to each other via a cable 210. A probe head 220 is provided at the head of the ultrasonic probe 200, and the main body of the ultrasonic measurement device 100 is provided with the display unit 300 that displays images. In FIG. 30C, the ultrasonic probe 220 is provided in the ultrasonic imaging device having the display unit 300. The ultrasonic imaging device of FIG. 30C can be realized by a general-purpose mobile information terminal such as a Smartphone, for example.

Note that the ultrasonic measurement device, the ultrasonic imaging device, or the like of the present embodiment may be realized by a program that performs a part or most part of processing. In this case, by a processor such as a CPU executing the program, the ultrasonic measurement device, the ultrasonic imaging device, or the like of the present embodiment is realized. Specifically, a program stored in a non-transitory information storage device is read, and the read program is executed by a processor such as a CPU. In this context, the information storage device (a computer readable device) is a device in which a program, data, and the like are stored, and whose functions can be realized by an optical disc (such as a DVD or CD), an HDD (hard disk drive), a memory (such as a card-type memory or a ROM), or the like. Also, a processor such as a CPU executes various types of processing of the present embodiment based on the programs (data) stored in the information storage device. That is, the information storage device has stored therein a program for causing a computer (device including an operation unit, a processing unit, a storage unit, and an output unit) to function as the components of the embodiments (programs for causing a computer to execute processing of the components).

Furthermore, the ultrasonic measurement device, the ultrasonic imaging device, and the like of the embodiments may include a processor and a memory. Here, the processor may be, for example, a CPU (Central Processing Unit). However, the processor is not limited to the CPU, and may employ various types of processors such as a GPU (Graphics Processing Unit) and a DSP (Digital Signal Processor). Furthermore, the processor may be a hardware circuit using an ASIC (Application Specific Integrated Circuit). Furthermore, the memory stores computer readable commands, and by the commands being executed by the processor, the components of the ultrasonic measurement device, the ultrasonic imaging device, or the like of the present embodiment will be realized. The memory in this context may be a semiconductor memory, such as an SRAM (Static Random Access Memory) or a DRAM (Dynamic Random Access Memory), a register, a hard disk, or the like. Furthermore, the commands in this context may be a command set of commands constituting the program, or commands to instruct the hardware circuit of the processor to operate.

As described above, the embodiments have been described in detail, but it can readily be appreciated to those skilled in the art that various modifications are possible without substantially departing from the novel features and effects of the invention. Therefore, all the modifications are included in the scope of the invention. For example, a term that is used in the specification and the drawings at least once together with a different term having a broader or the same meaning can be replaced with this different term in any place of the specification or the drawings. Furthermore, the configurations and operations of the ultrasonic measurement device and the ultrasonic imaging device are not limited to those described in the present embodiment, and various modifications are possible.

The entire disclosure of Japanese Patent Application No. 2014-222471 filed on Oct. 31, 2014 is expressly incorporated by reference herein.

What is claimed is:

1. An ultrasonic measurement device comprising:
a pulse signal output circuit that outputs a pulse signal based on a clock signal; and
a resonance circuit that is connected to the pulse signal output circuit, includes an ultrasonic transducer element, and has frequency characteristics of a low-pass filter,
wherein the pulse signal output circuit outputs a plurality of pulse signals that are different from each other in at least one of pulse signal voltage, pulse signal width, and pulse output timing.

2. The ultrasonic measurement device according to claim 1,
wherein the pulse signal output circuit outputs a first pulse signal having a first pulse voltage at a first pulse output timing, and
outputs a second pulse signal having a second pulse voltage, which is different from the first pulse voltage, at a second pulse output timing after the first pulse output timing.

3. The ultrasonic measurement device according to claim 2,
wherein the first pulse signal is a first polarity pulse signal having one of the positive polarity and the negative polarity, and the second pulse signal is a second polarity pulse signal having the other different polarity, and
the absolute value of the second pulse voltage is smaller than the absolute value of the first pulse voltage.

4. The ultrasonic measurement device according to claim 1,
wherein the pulse signal output circuit outputs a first pulse signal having a first pulse width at the first pulse output timing, and outputs a second pulse signal having a second pulse width, which is different from the first pulse width, at the second pulse output timing after the first pulse output timing.

5. The ultrasonic measurement device according to claim 4,
wherein the second pulse width is greater than the first pulse width.

6. The ultrasonic measurement device according to claim 2,
wherein the second pulse signal is a pulse signal for suppressing resonant vibration of a transmission signal to the ultrasonic transducer element.

7. The ultrasonic measurement device according to claim 2,
wherein the second pulse signal is a pulse signal for suppressing sound reverberation of a transmission signal to the ultrasonic transducer element.

8. The ultrasonic measurement device according to claim 1,
wherein the pulse signal output circuit outputs the first pulse signal having the first pulse voltage and the first pulse width at the first pulse output timing, and
outputs, at the second pulse output timing after the first pulse output timing, the second pulse signal having the second pulse voltage whose absolute value is smaller than that of the first pulse voltage and having the second pulse width, which is greater than the first pulse width.

9. The ultrasonic measurement device according to claim 1,
wherein the pulse signal output circuit outputs one or more first time period pulse signals during a first time period, outputs no pulse signals during a second time period after the first time period, and outputs a third time period pulse signal during a third time period after the second time period.

10. The ultrasonic measurement device according to claim 9,
wherein the pulse signal output circuit outputs a pulse signal for suppressing sound reverberation of a transmission signal to the ultrasonic transducer element during the third time period.

11. The ultrasonic measurement device according to claim 1,
wherein the pulse signal output circuit outputs a pulse signal that causes an envelope curve of a waveform of a transmission signal to the ultrasonic transducer element to have the shape of a sine wave.

12. An ultrasonic imaging device comprising:
the ultrasonic measurement device according to claim 1, and
a display unit that displays image data for display that is created based on an ultrasonic echo in response to a transmitted ultrasonic wave.

13. An ultrasonic imaging device comprising:
the ultrasonic measurement device according to claim 2, and
a display unit that displays image data for display that is created based on an ultrasonic echo in response to a transmitted ultrasonic wave.

14. An ultrasonic imaging device comprising:
the ultrasonic measurement device according to claim 3, and
a display unit that displays image data for display that is created based on an ultrasonic echo in response to a transmitted ultrasonic wave.

15. An ultrasonic imaging device comprising:
the ultrasonic measurement device according to claim 4, and
a display unit that displays image data for display that is created based on an ultrasonic echo in response to a transmitted ultrasonic wave.

16. An ultrasonic imaging device comprising:
the ultrasonic measurement device according to claim 5, and
a display unit that displays image data for display that is created based on an ultrasonic echo in response to a transmitted ultrasonic wave.

17. An ultrasonic imaging device comprising:
the ultrasonic measurement device according to claim 6, and
a display unit that displays image data for display that is created based on an ultrasonic echo in response to a transmitted ultrasonic wave.

18. An ultrasonic imaging device comprising:
the ultrasonic measurement device according to claim 7, and
a display unit that displays image data for display that is created based on an ultrasonic echo in response to a transmitted ultrasonic wave.

19. An ultrasonic imaging device comprising:
the ultrasonic measurement device according to claim 8, and
a display unit that displays image data for display that is created based on an ultrasonic echo in response to a transmitted ultrasonic wave.

20. An ultrasonic imaging device comprising:
the ultrasonic measurement device according to claim 9, and
a display unit that displays image data for display that is created based on an ultrasonic echo in response to a transmitted ultrasonic wave.
